(19) United States
(12) Patent Application Publication
(10) Pub. No.: US 2025/0283800 A1
(43) Pub. Date: Sep. 11, 2025

(54) PROBES FOR FLUORESCENCE IMAGING

(71) Applicant: The General Hospital Corporation, Boston, MA (US)

(72) Inventors: Ralph Weissleder, Peabody, MA (US); Elias Arturo Halabi Rosillo, Cambridge, MA (US)

(21) Appl. No.: 18/844,896

(22) PCT Filed: Mar. 31, 2023
(86) PCT No.: PCT/US2023/017220

§ 371 Date: Sep. 6, 2024

Related U.S. Application Data
(60) Provisional application No. 63/326,837, filed on Apr. 2, 2022.

(51) Int. Cl. G01N 15/1434; C09B 62/473; C09K 11/06; G01N 15/01; G01N 15/10

(52) U.S. Cl. CPC G01N 15/1434; C09B 62/473; C09K 11/06; G01N 15/01; C09K 2211/1007; C09K 2211/1014; C09K 2211/1018; G01N 2015/1006

(57) ABSTRACT

The present disclosure provides photocleavable rhodamine probes that facilitate live- and fixed-cell immunofluorescence. The ultra-fast spirocyclization of the dye following cleavage depletes the fluorescence signal, enabling cyclic multiplexed imaging.

### CLAIM OF PRIORITY

This application claims priority to U.S. Provisional Patent Application Ser. No. 63/326,837, filed on Apr. 2, 2022, the entire contents of which are hereby incorporated by reference.

### TECHNICAL FIELD

This invention relates to photocleavable rhodamine probes that facilitate live- and fixed-cell immunofluorescence. In particular, this disclosure provides a dye that undergoes ultra-fast spirocyclization following cleavage or a photo-immolating triazene linker. The spirocyclic product depletes the fluorescence signal, enabling cyclic multiplexed imaging.

### BACKGROUND

Processing cellular samples, e.g., for immunostaining and image cytometry, often can be quite challenging. One drawback is that the cellular samples are often scant (often <1,000 cells per pass from a fine needle aspirate), limiting the number of special stains that can be done, and also delicate, lacking the structural scaffold of intact tissue architecture. Even when processed with fluorescent antibodies, the number of different stains for cellular samples is typically limited to 4-6 and is often insufficient for in depth cancer cell profiling for diagnosis or treatment assessment. This limitation also extends to immune profiling, where significantly more than 4-6 markers need to be interrogated so that analysis reflects the representative immunocyte populations in the tumor microenvironment.

### SUMMARY

Highly multiplexed, cyclic fluorescence imaging has advanced the scientists' understanding of the biology, evolution, and complexity of human diseases. Currently available cyclic methods still have considerable limitations including the need for long quenching times and extensive wash steps. The present disclosure advantageously provides fluorochrome compounds (e.g., rhodamines) that can be efficiently inactivated by a single light pulse (about 405 nm) by means of a photo-immolating triazene linker. Upon UV-light irradiation, the rhodamines are cleaved off from the antibody conjugates and undergo a fast intramolecular spirocyclization that inherently switches off their fluorescence emission without the need to wash or add exogenous chemicals. The data provided in this disclosure shows that the “switch-off” probes within the instant claims are advantageously fast (<4s), highly controllable, biocompatible, and allow spatiotemporal quenching control of live and fixed samples.

In one general aspect, the present disclosure provides a compound of Formula (I):

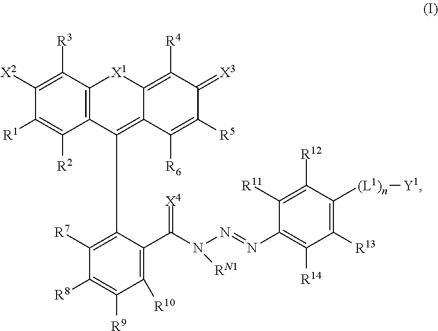

or a pharmaceutically acceptable salt thereof

In another general aspect, the present disclosure provides a protein conjugate of Formula (II):

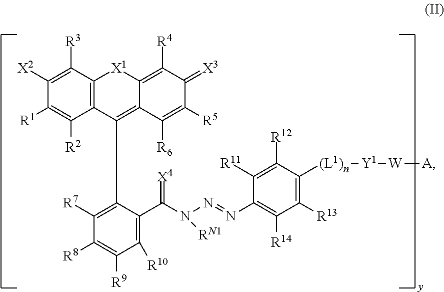

or a pharmaceutically acceptable salt thereof.

In yet another general aspect, the present disclosure provides a method of examining a cell or a component of a cell, the method comprising:
(i) contacting the cell with a protein conjugate of Formula (II), or a pharmaceutically acceptable salt thereof, or a composition of same;
(ii) imaging the cell with an imaging technique; and
(iii) after (ii), contacting the cell with a light of a wavelength.

In yet another general aspect, the present disclosure provides a method of profiling a cell,

In yet another general aspect, the present disclosure provides a method of examining a cell using a cytometry technique, the method comprising (i) obtaining a cell from the subject; and (ii) examining the cell according to the imaging method as disclosed herein.

In yet another general aspect, the present disclosure provides a method of diagnosing a disease or condition of a subject by examining pathology of a cell obtained from the subject, the method comprising (i) obtaining a cell from the subject; and (ii) examining the cell according to the imaging method as disclosed herein.

In yet another general aspect, the present disclosure provides a method of monitoring progression of disease or condition of a subject by examining pathology of a cell obtained from the subject, the method comprising (i) obtaining a cell from the subject; and (ii) examining the cell according to the imaging method as disclosed herein.

In yet another general aspect, the present disclosure provides a method of detecting a disease biomarker in a cell, the method comprising (i) obtaining a cell from the subject; and (ii) examining the cell according to the imaging method as disclosed herein.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1. Switch-off concept of FLASH-off probes. A. Structure of FLASH-off 550 antibody conjugate and formation of photoproducts. B. Workflow for FLASH-off staining of immune cells based on rapid, “on-stage” light-based quenching and cyclic multiplexed imaging enabling spatial analysis of complex cells and tissues.

FIG. 2. Synthesis of FLASH-off probes. A. Three-step synthesis route to obtain compounds 1a-d starting from parent rhodamine scaffolds 2a-d (top). Selected rhodamine scaffolds investigated in this study with an absorbance range of (500-650 nm, bottom). B. Synthesis of the triazene photo-cleavable linker 3. C. Synthesis of the PEG4 linker 6.

FIG. 3. Solution experiments and FLASH-off mechanism. A. Scheme depicting the photolysis of probe 1b yields exclusively the formation of the open and closed photoproducts of the methyl xanthamide 7b at different pH. B. LC/MS chromatographs of solutions of compound 1b before and after photolysis (1 min) at pH=3 (top, citrate buffer) and pH=8 (phosphate buffer) using UV lamp (405 nm LED, power 1.4 mW cm⁻²). C. Mass spectra (ES−) of peaks with m/z corresponding to compound 1b, and the open and closed forms of 7b. D. pH dependence of spirocyclization upon triazene cleavage. At physiologic pH and before photo-cleavage, the FLASH-off probes are bright and detectable during imaging. After photo-cleavage, the non-fluorescent versions are highly favored (about 166-fold reduction in fluorescent signal intensity). E. Pictures of vials containing solutions of compounds 1a-d before (left), during (middle), and after (right) irradiation (1.14 mW cm⁻²) at pH=8.

FIG. 4. Live-cell imaging and cycling. A. Cell viability assay determined for A431 cells incubated with FLASH-off 600 (0.15-40 μM, 72 h). The graph shows no apparent toxicity at imaging concentrations (about 0.1 μM) used for live cells. Subtle cellular toxicity was observed at >100× of the imaging contraption with an IC50 of ˜40 μM. B. Live-cell quenching experiments using anti-Cetuximab-FLASH-off 647 (657 nm channel, 6.9 mW cm-2) and line profile quenching (10 s, 405 nm 660 channel 660 μW cm⁻²). C. Cyclic imaging using anti-EGFR-FLASH-off 550 (550 nm channel, 20.3 mW cm⁻²) followed by anti-EGFR2-FLASH-off 647 (647 nm channel, 6.9 mW cm⁻²) demonstrates excellent fluorescence quenching (<90%) and confirms no loss of antibody function during sequential staining using FLASH-off antibody conjugates. Scale bars in B=20 μm and in C=5 μm.

FIG. 5. Tissue imaging. A. Multiplexed tissue imaging in FFPE tonsil sections with anti-PD-1, CD11b, CD45 and CK FLASH-off (550 and 647 nm channel, 20.3 mW cm⁻² and 6.9 mW cm⁻²) conjugates and using DAPI (405 nm channel, 660 μW cm⁻²) and anti-CD14-MB488 (488 nm channel, 6.65 mW cm-2) as non-photoquenchable control stains (left). Reconstruction of a 6-color merged image was achieved by merging the single channels (middle). The image after photoquenching (10 s, 405 nm 660 channel 660 μW cm⁻², right) depicts the residual signal from the non-photoquenchable controls. B. Selective photobleaching to demonstrate local quenching control writing “CSB” on a tonsil section stained with anti-CD45-FLASH-off 550 (550 nm channel, 6.9 mW cm⁻²) and anti-CD45-FLASH-off 650. The signal could be recovered by subsequent re-staining cycles. Scale bars in A=40 μm, B=25 μm and C=120 μm.

FIG. 6 List of antibodies used for FLASH-off conjugation and immune cell profiling.

FIG. 7 Summary of probes used in this study.

FIG. 8 Photophysical properties of dyes.

FIG. 9. Proof-of-principle for developing ON→OFF xanthene dyes. A.

Chemical structures of parent C2′-carboxy xanthene, di-substituted xanthamides and mono-substituted xanthenes (e.g., methyl xanthamides). B. pH profiles showing absorbance and emission for each compound shown as fluorescein derivatives. These results indicate that di-substituted xanthenes cannot undergo spirocylization and thus are locked in a fluorescent state regardless of pH. The methyl xanthamide derivative, highly favors the non-emissive spirocyclic form. Equilibrium factors are strongly dependent on the electron donor-acceptor groups (D) and heteroatom in the xanthene scaffold.

FIG. 10. Synthesis of methyl xanthamide photoproducts. Probes 7a-d were synthesized by direct coupling of parent rhodamines 2a-d and methylamine (organic synthesis, general procedure 4).

FIG. 11. Normalized emission spectra of compounds 1a-d . Fluorescence intensity scans were performed in citric acid+Na₂HPO₄ buffer at pH=8 (10 μM, n=3) and normalized to the λmax of emission for each compound (n=3).

FIG. 12. Ph dependency on fluorescent emission of compounds 1a-d . A pH profile of each probe was determined in citric acid+Na₂HPO₄ buffer at pH=3, 4, 5, 6, 7, 8 and 9 (10 μM, n=3). The fluorescence intensity scan was integrated and normalized to the highest value for each compound independently. Fluorescent signal (>75%) was retained for all compounds regardless of change in pH.

FIG. 13. Photolysis of 1a-d and yield of photoproducts. A. Liquid chromatograms of solutions containing probes 1a-d (10 μM) before (black solid line) and after irradiation (405 nm LED, 1 min, 1.14 mW cm⁻²), depicted in a colored line for each compound. B. Mass spectrometry analysis of the photoproducts detected for each peak in panel A. Photoproducts were identified by their mass-to-charge ratio (m/z) and their retention times were also compared to synthesized methyl xanthamides 7a-d (data not shown). C. The yield of the photoproducts was estimated by integrating the area under each peak of the chromatogram in panel A (excluding the injection peak).

FIG. 14. Proposed mechanism for the photo-cleavage of FLASH-off probes. A. Solvent and pH-independent component depicting compound 1c undergoing loss of N₂ gas and formation of the methyl xanthamide 7c. B. Liquid chromatogram showing the pH-dependency for forming the open and closed photoproducts 7c when irradiations are performed at pH=3 vs. pH=8 (1 min, 150 ρW cm⁻²). Mass spectrometry analysis of the linker photoproduct detecting the phenol 8, anisole 9, and phenyl 12 adducts when the solutions of compound 1c were irradiated in water, methanol, and acetonitrile respectively (1 min, 150 ρW cm⁻²).

FIG. 15. Kinetics of photolysis. The half-life of compounds 1a-d was determined by plotting the percent of compound 1a-d present in solutions (100 μM) before irradiation (t=0 min) and after irradiation (t=2, 4, 6, 8, 10, 15, 20, 25, 30, 40, 50 and 60 s) using 405 nm light (LED, 1.14 mW cm⁻²) at pH=3. The area under the curve was integrated from the liquid chromatograms and normalized to the initial concentration (t=0 min). Under constant light irradiations, all solutions containing compounds 1a-d reached completion in between 30-60 s (n=3).

FIG. 16. Spectroscopic studies of photo reactions for different FLASH-off probes. A. Schematic depiction of the photo reactions for FLASH-off 500-650 (1a-d respectively) with the absorbance and emission spectra scans of the photo reactions (5 μM) in (citric acid+Na₂HPO₄ buffer, pH=8) before (red) and after (dark) irradiation 1 min 365 nm lamp. All spectra were taken in triplicates. B. Pictures of solutions of panel A in white light (top and bottom) and under UV light (middle).

FIG. 17. Stability of FLASH-off probes in PBS. A. Schematic depiction of FLASH-off 650 hydrolysis in PBS at 4° C. for 30 days. B. Normalized LC chromatograms and C. ES⁻ chromatograph confirming the integrity of FLASH-off 650 1d (ES⁻ chromatograph) with no detected hydrolysis to the corresponding methyl xanthamide 7d.

FIG. 18. Fixed-splenocytes imaging and cycling. Fixed splenocytes stained with anti-MHCII-FLASH-off 550 and anti-MHCII-FLASH-off 650 (˜5 μg antibody/mL, DOL 3.5 and 2) and imaged in the 550 nm (top, 20.3 mW cm⁻²) and 647 nm (bottom, 6.9 mW cm⁻²) imaging channels. The region of interest (ROIs) are depicted as red-dashed squares showing a zoomed-in area with an average of 41.88 and 32.3% positively stained cells for FLASH-off 550 and FLASH-off 650 antibody conjugates respectively. C. Quenching of the signal (>92% and >98%) was determined by normalizing the signal before and after irradiation with 10 s (405 nm, normalized intensity after 10 s of irradiation (660 μW cm⁻²). All images have the same scale bar=75 μm.

FIG. 19. Quenching kinetics for FLASH-off 550 and FLASH-off 650. A. Fixed splenocytes stained with MHCII-FLASH-off 650 and MHCII-FLASH-off 550 (not shown) to show the strategy for quantifying the efficiency of photoquenching under constant 405 nm laser irradiation with varying laser power (10-15%=80 and 120 W cm⁻²). B. Plots showing the results of the experiment presented in panel A. The integrated density from the ROI was plotted against time and the half-life (t_{1/2}) for FLASH-off 550 and FLASH-off 650 were 21.5 s and 7.15 s respectively at 80 ρW cm⁻² (n=3). Scale bars in A=100 μm and B=20 μm.

FIG. 20. Cyclic staining in fixed splenocytes. Fixed splenocytes stained with antibody conjugates antiCD45-FLASH-off 550, antiMHCII-FLASH-off 550 and antiCD4-FLASH-off 650 and antiCD8-FLASH-off 650 (45 min, ˜5 μg antibody/mL, DOL=3.0, 3.5, 4.0, 1 respectively). DAPI was used as a nuclear staining (bottom left). Percentages of stain cells over total cells was determined by colocalization of the signal and particle analysis (ComDET) and yielded 79%, 46%, 14% and 2% for CD45, MCHII, CD4 and CD8 cells respectively. All images have the same scale bar=20 m.

FIG. 21. Comparison of photoquenching efficiency in fixed splenocytes. Cells were stained with A. MHCII-FLASH-off 550 (top) and CD45-FLASH-off 550 (bottom) and B. MHCII-FAST647 (top) and CD45-FAST647 (bottom). These images show that upon 4s 405 nm light irradiation, no quenching was evident in the non-photoquenchable control. All images have the same scale bar=20 m. All experiments were done in triplicates (n=3).

FIG. 22. Live-cell quenching experiments. A. Live A431 cell experiments using Cetuximab-FLASH-off 647 and quenched (4s, 405 nm channel) with no washing steps. B. Zoomed in ROI of panel A showing selective staining of the membrane. C. Line profile indicating the efficient quenching effect after irradiation. Scale bars A & C=10 m, B=5 μm (n=3).

FIG. 23. Quenching can be spatially controlled. A. Focused local quenching experiment (top) in tonsil tissue sections using an epifluorescent microscope. Local quenching was performed using a 40× magnification (DAPI, 8 s) and read-out was performed with 10× magnification (647 nm channel). (<90%) of fixed human tonsil stained with CD45-FLASH-off 650. Whole sample quenching (bottom) was achieved by soaking the sample with buffer at pH=8.4 and using an external 405 nm lamp placed at about 1 cm distance for 2 min. Scale bar=50 m (top), 120 m (bottom).

### DETAILED DESCRIPTION

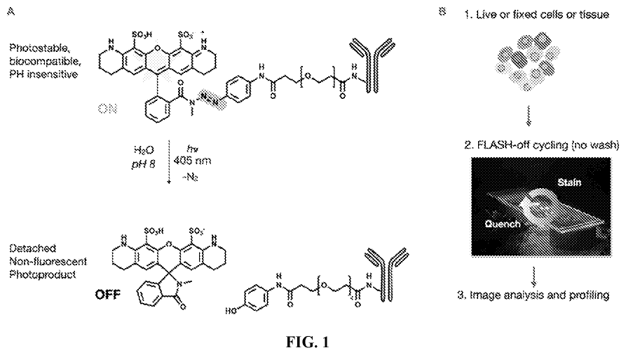

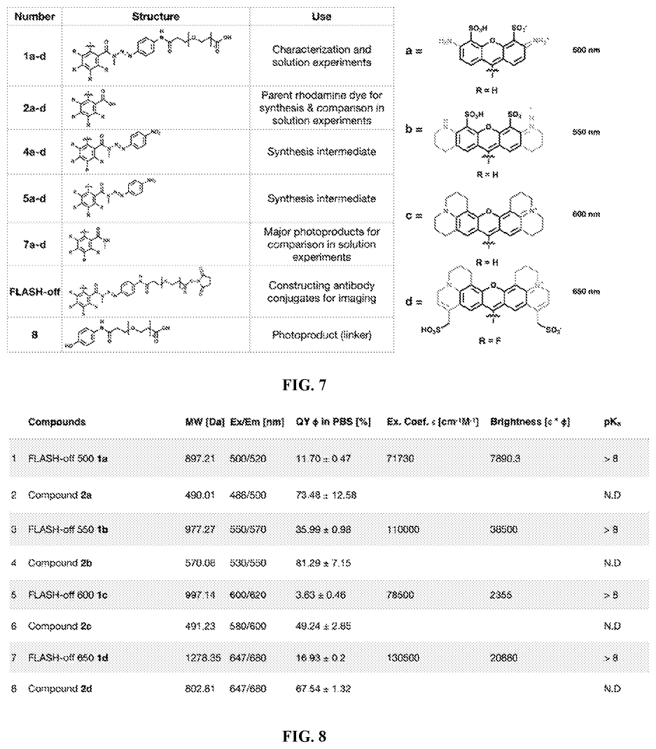

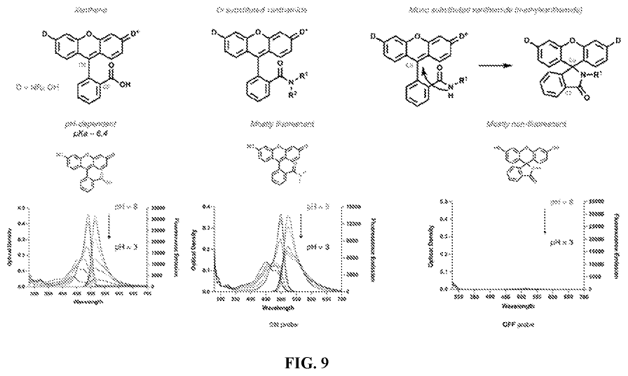

Multiplexed sensing strategies have revolutionized modern biology by allowing single-cell profiling, spatial transriptomics and most recently temporal transciptomics. Spatial and temporal profiling technologies are largely based on cyclic imaging where tissue and cells are stained with multiple affinity ligands, then quenched and re-stained followed by additional imaging cycles. The most common chemical quenching approaches use hydrogen peroxide, NaOH, formamide or tris(2-carboxyethyl)phosphine (TCEP). A number of different technologies based on DNA barcoding or bioorthogonal quenching have also been recently reported. The latter techniques are generally gentler on the sample and have reduced cycling time from days to hours or minutes. Irrespective of the specific approach, chemical means of quenching require liquid handling and manipulation of the specimen between cycles resulting in registration challenges. In order to minimize specimen handling steps and to speed up cycling, the present disclosure advantageously provides fluorochromes that can be rapidly and controllably inactivated by a selective light pulse without causing interference with other imaging channels. The development of photo-activatable switch-off fluorochromes has been much more challenging compared to designing switch-on fluorescent reporters. Reasons for it are that conventional photo-responsive linkers can cause significant fluorescence quenching of the initial fluorescent state of the fluorophore and enhance aggregation in aqueous media. A key challenge has been thus to identify physiologically compatible and stable groups that do not affect the spectral properties of the dye in the visible range while responding to a selected wavelength of light to yield a non-fluorescent photoproduct. A vast range of photo-responsive functional groups have been reported, such as i) nitrobenzyl, ii) arylcarbonylmethyl, ii) polyaromatic perylene, iii) coumarin-4-ylmethyl iv) diazoindanones and iv) triazenes, a less studied class of photo-reactive compounds. In contrast to other side groups, linear aromatic triazenes do not undergo fluorescence emission and can be chemically modified to fine-tune their photo-release kinetics, resistance toward hydrolysis at physiological pH, and solubility in water. Without being bound by any theory or speculation, the present disclosure provides switch-off xanthenes with a photo-responsive linear triazene that drives the innate equilibrium between the open, highly fluorescent, and the closed, non-fluorescent form. To switch off the fluorescent signal, a required intramolecular rearrangement, elicited by the photo-cleavage of the triazene, would interrupt the conjugation system of the fluorophore and terminate its fluorescence emission. The well-understood spirocyclic equilibrium is highly dependent on factors including pH, solvent, donor-acceptor groups, and the substituents present at the 2′-position of the pendant aromatic ring (C-9′-atom of the xanthene core, FIG. 9). Substituting the carboxylate with an amide, for instance, has been demonstrated to significantly favor the non-fluorescent spirocyclic form at physiological pH. Compounds within the present claims advantageously include linear alkyl triazene (e.g., methyl triazene) moiety at the aforementioned position. Without being bound by a speculation, this moiety undergoes photolytic cleavage to release alkyl xanthamides (e.g., methyl xanthamides) as photoproducts (FIG. 1). The present disclosure also provides a straightforward synthetic route to convert different rhodamine scaffolds into fast light-activatable switch (FLASH)-off probes within the present claims, such as compounds 1a-d (FIG. 7). Certain embodiments of inactivatable fluorophore compounds, their biocompatible conjugates, as well as the methods of using these compounds and/or conjugates for imaging, e.g., live or fixed cells, are described herein. Advantageously, because the probes within the present claims can be turned off by light rather than by chemical means, the compounds and methods of this disclosure provide extraordinary spatiotemporal control and unprecedented localized quenching.

### Reagents and Linkers

In some embodiments, the present application provides a compound of Formula (I):

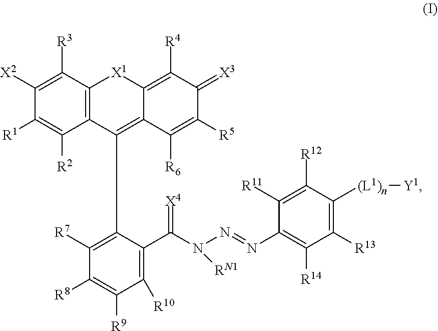

or a pharmaceutically acceptable salt thereof, wherein:
X¹ is selected from O, S, C(R¹⁵)₂, and Si(R¹⁵)₂;
each R¹⁵ is independently selected from H, C₁₋₆ alkyl, C₂₋₆ alkenylene, and C₁₋₆ haloalkyl; wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
or any two R¹⁵ together with the C or Si atom to which they are attached from a 3-7 membered saturated or unsaturated carbocyclic or heterocyclic ring, which is optionally substituted with 1 or 2 substituents independently selected from halo OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy;
R^{N1} is selected from C₁₋₃ alkyl and C₁₋₃ haloalkyl, wherein said C₁₋₃ alkyl is optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
X² is selected from OR^{N2} and N(R^{N2})₂;
X³ is selected from 0 and NR^{N2};
X⁴ is 0, S; or NR^{N2};
each R^{N2} is independently selected from H, C₁₋₆ alkyl, and C₁₋₆ haloalkyl, wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
or any two R^{N2} together with the O or N atom to which they are attached from a 3-7 membered saturated or unsaturated carbocyclic or heterocyclic ring, which is optionally substituted with 1 or 2 substituents independently selected from halo, OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy;
R¹, R², R³, R⁴, R⁵, R⁶, R⁷, R⁸, R⁹, R¹⁰, R¹¹, R¹², R¹³, and R¹⁴ are each independently selected from H, halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C₁₋₆ alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃ haloalkoxy;
or each pair of R^{N2} and R³, R^{N2} and R¹, R^{N2} and R⁴, R^{N2} and R⁵, R¹ and R², and R⁵ and R⁶, together with the C, N, or O atoms to which they are attached, form a 5-8 membered saturated or unsaturated carbocyclic or heterocyclic ring, which is optionally substituted with 1, 2, or 3 substituents independently selected from halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C₁₋₆ alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃ haloalkoxy;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each R^{N} is independently selected from H, C₁₋₃ alkyl, and C₁₋₃ haloalkyl;
each x is independently an integer from 1 to 2,000;
Y¹ is selected from NR^{N2}R^{1A} O^{2A}, C(═O)OR^{3A}, and a group reactive with a side chain of an amino acid of a protein.
R^{1A} selected from H and an amine protecting group;
R^{2A} is selected from H and an alcohol protecting group; and
R^{2A} is selected from H and a carboxylic acid protecting group.

In some embodiments, X¹ is O. In some embodiments, X¹ is S. In some embodiments, X¹ is C(R¹⁵). In some embodiments, X¹ is Si(R¹⁵)₂.

In some embodiments, R¹⁵ is selected from H, C₁₋₆ alkyl, C₂₋₆ alkenylene, and C₁₋₆ haloalkyl. In some embodiments, any two R¹⁵ together with the C or Si atom to which they are attached from a 3-7 membered saturated or unsaturated carbocyclic or heterocyclic ring, which is optionally substituted with 1 or 2 substituents independently selected from halo OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy. In some embodiments, any two R¹⁵ together with the C or Si atom to which they are attached from a 3-7 membered saturated or unsaturated carbocyclic or heterocyclic ring, which is optionally substituted halo OH, CN, C₁₋₃ alkoxy, or C₁₋₃ haloalkoxy.

In some embodiments, R^{N1} is selected from C₁₋₃ alkyl and C₁₋₃ haloalkyl. In some embodiments, R^{N1} IS C₁₋₃ alkyl. In some embodiments, R^{N1} is C₁₋₃ haloalkyl. In some embodiments, R^{N1} is H. In some embodiments, R^{N1} is methyl, ethyl, propyl, or isopropyl. In some embodiments, R^{N1} IS C₁₋₃ alkyl, substituted with OH, NH₂, C₁₋₃ alkylamino, di(C₁₋₃ alkyl)amino, C₁₋₃ alkoxy, or C₁₋₃ haloalkoxy.

In some embodiments, X² is OR^{N2}. In some embodiments, X² is N(R^{N2})₂. In some embodiments, X³ is O. In some embodiments, X³ is NR^{N2}. In some embodiments, X⁴ is O. In some embodiments, X⁴ is NR^{N2}. In some embodiments, X⁴ is S.

In some embodiments, R^{N2} is selected from H, C₁₋₆ alkyl, and C₁₋₆ haloalkyl, wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy. In some embodiments, R^{N2} is selected from H, C₁₋₆ alkyl, and C₁₋₆ haloalkyl. In some embodiments, R^{N2} is selected from H and C₁₋₃ alkyl.

In some embodiments, X² is N(R^{N2})₂, and any two R^{N2} together with the N atom to which they are attached from a 3-7 membered saturated or unsaturated carbocyclic or heterocyclic ring, which is optionally substituted with 1 or 2 substituents independently selected from halo, OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy. In some embodiments, the ring is heterocyclic

In some embodiments, R¹, R², R³, R⁴, R⁵, R⁶, R⁷, R⁸, R⁹, R¹⁰, R¹¹, R¹², R¹³, and R¹⁴ are each independently selected from H, halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, and di(C₁₋₆ alkyl)amino.

In some embodiments, a pair of R^{N2} and R³, R^{N2} and R¹, R^{N2} and R⁴, R^{N2} and R⁵, R¹ and R², and/or R⁵ and R⁶, together with the C, N, or O atoms to which they are attached, form a 5-8 membered saturated or unsaturated carbocyclic or heterocyclic ring, which is optionally substituted with 1, 2, or 3 substituents independently selected from halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C₁₋₆ alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃ haloalkoxy.

In some embodiments, n is an integer from 1 to 7. In some embodiments, n is an integer from 1 to 5. In some embodiments, n is 1. In some embodiments, n is 2. In some embodiments, n is 3.

In some embodiments, each L¹ is independently selected from NH, O, C(═O), C₁₋₆ alkylene, —(OCH₂CH₂)ₓ—, and —(CH₂CH₂O)ₓ—. In some embodiments, n is an integer from 1 to 5, and each L¹ is selected from NH, O, C(═O), and C₁₋₆ alkylene. In some embodiments, n is 1 and L¹ is C₁₋₆ alkylene. In some embodiments, R^{N} is H. In some embodiments, R^{N} IS C₁₋₃ alkyl. In some embodiments, x is an integer from 2 to 10. In some embodiments, x is 3, 4, 5, or 6.

In some embodiments, (L)ₙ comprises a side chain of an amino acid (e.g., lysine, serine, threonine, cysteine, tyrosine, aspartic acid, or glutamic acid), and Y¹ comprises the terminal functional group of the side chain of the amino acid (e.g., N, O, S, or C(═O)).

In some embodiments, Y¹ is NHR^{1A}. In some embodiments, R^{1A} is an amine protecting group. In some embodiments, Y¹ is NH₂. In some embodiments, Y¹ is OR^{2A}. In some embodiments, Y¹ is OH. In some embodiments, R^{2A} is an alcohol protecting group. In some embodiments, Y¹ is C(═O)R^{3A}. In some embodiments, Y¹ is C(═O)OH.

In some embodiments, Y¹ is a group reactive with a side chain of an amino acid of a protein. In some embodiments, the group reactive with a side chain of an amino acid of a protein is an activated ester group.

In some embodiments, the compound of Formula (I) has formula:

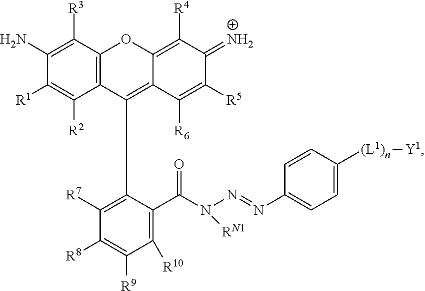

or a pharmaceutically acceptable salt thereof.

In some embodiments:
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino,
R⁷, R⁸, R⁹, and R¹⁰ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino; and
R^{N1} is selected from C₁₋₃ alkyl and C₁₋₃ haloalkyl.

In some embodiments:
R¹, R², R³, R⁴, R⁵, and R⁶ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy, wherein said C₁₋₃ alkyl is optionally substituted with OH, SO₃H, C(═O)OH, CN, C₁₋₃ alkoxy, and C₁₋₃ haloalkoxy;
R⁷, R⁸, R⁹, and R¹⁰ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy; and
R^{N1} is C₁₋₃ alkyl.

In some embodiments, the compound of Formula (I) has formula:

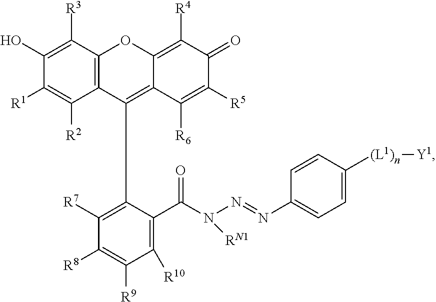

or a pharmaceutically acceptable salt thereof.

In some embodiments:
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino,
R⁷, R⁸, R⁹, and R¹⁰ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino; and
R^{N1} is selected from C₁₋₃ alkyl and C₁₋₃ haloalkyl.

In some embodiments:
R¹, R², R³, R⁴, R⁵, and R⁶ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy, wherein said C₁₋₃ alkyl is optionally substituted with OH, SO₃H, C(═O)OH, CN, C₁₋₃ alkoxy, and C₁₋₃ haloalkoxy;
R⁷, R⁸, R⁹, and R¹⁰ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy; and
R^{N1} is C₁₋₃ alkyl.

In some embodiments, n is an integer from 1 to 5, and each L¹ is selected from NH, O, C(═O), and C₁₋₆ alkylene.

In some embodiments, the compound of Formula (I) is selected from any one of the following compounds:

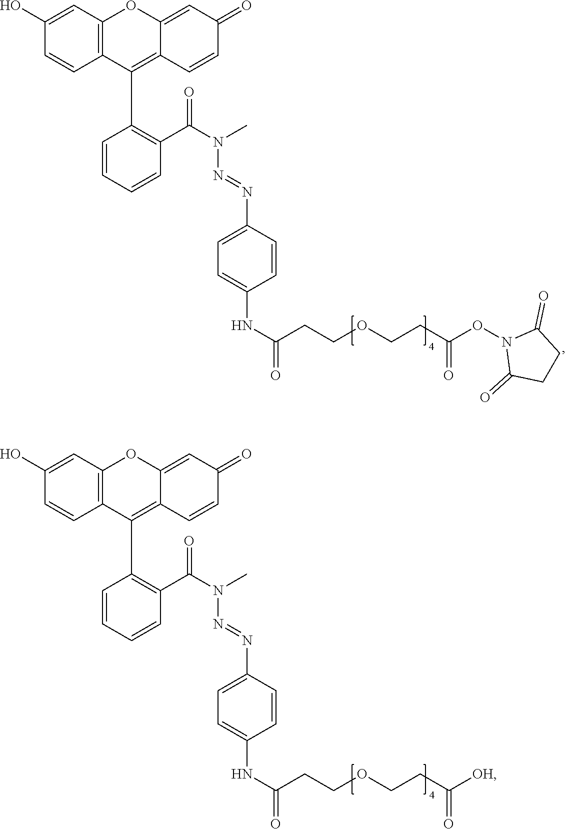

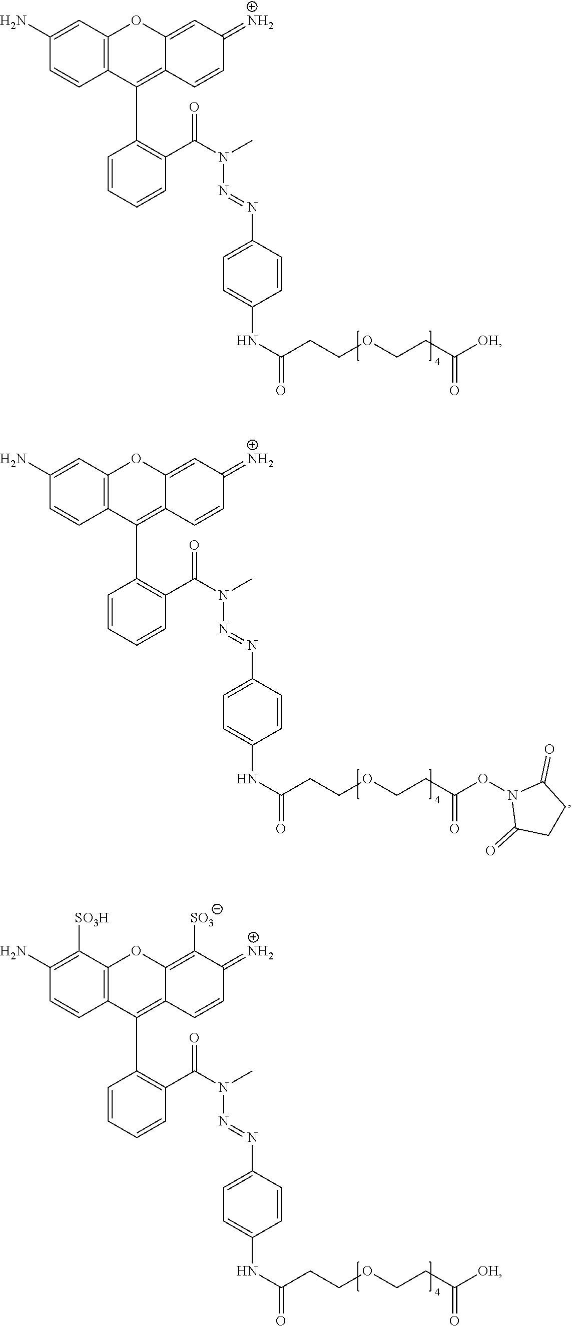

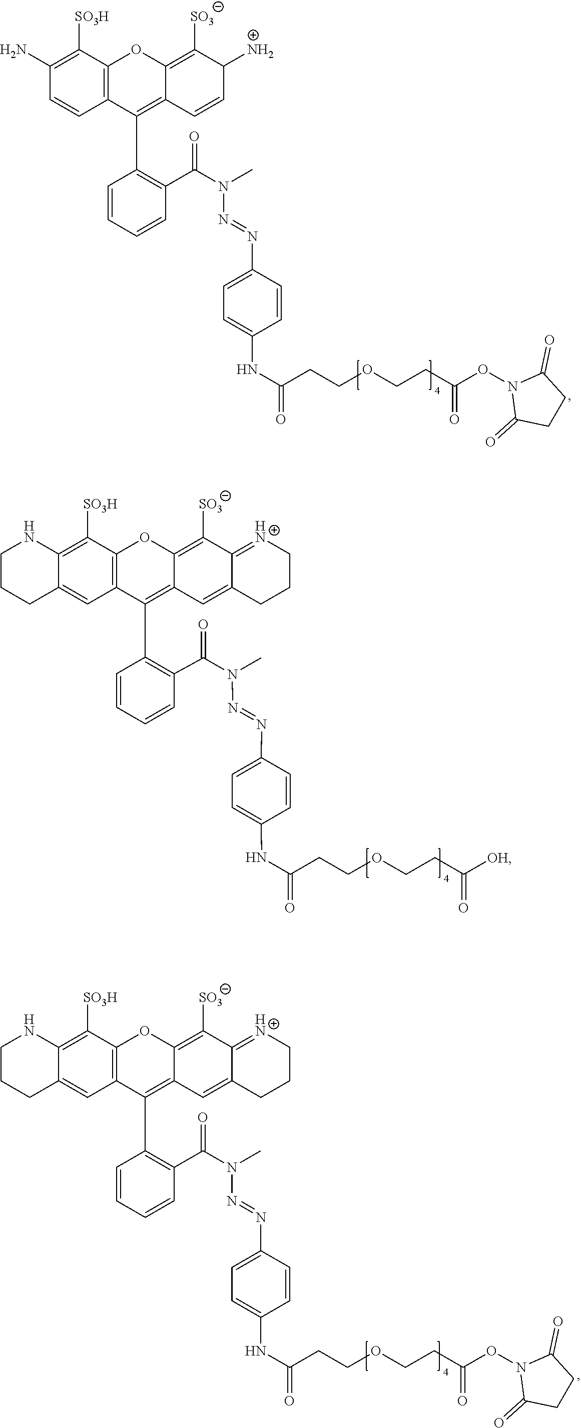

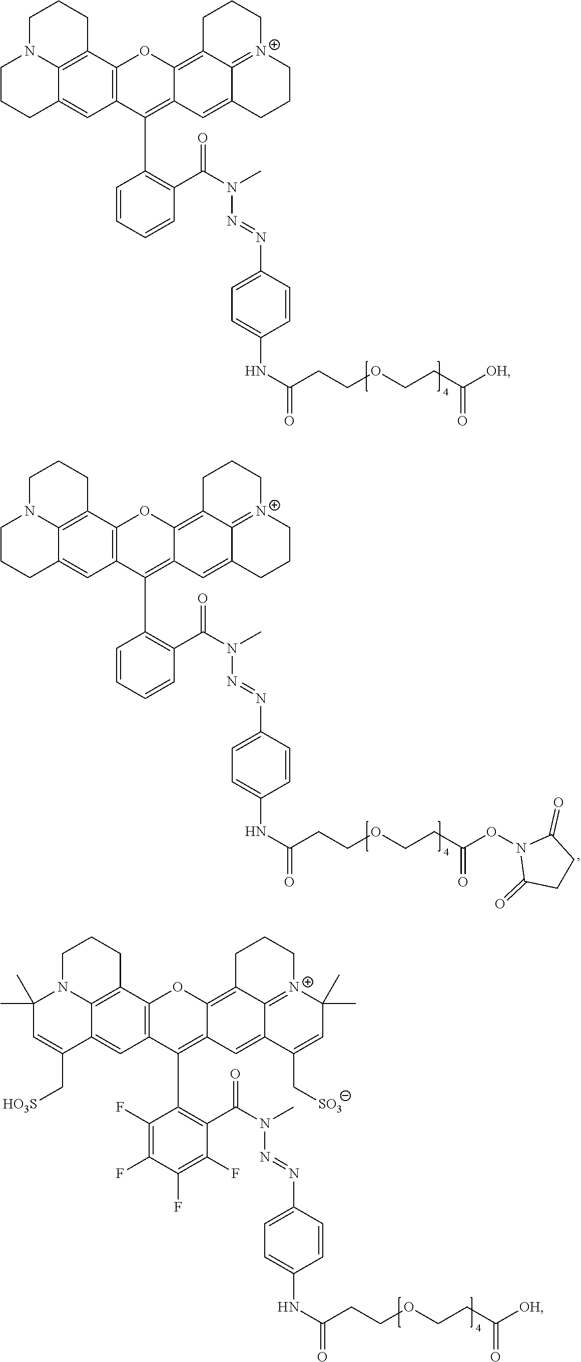

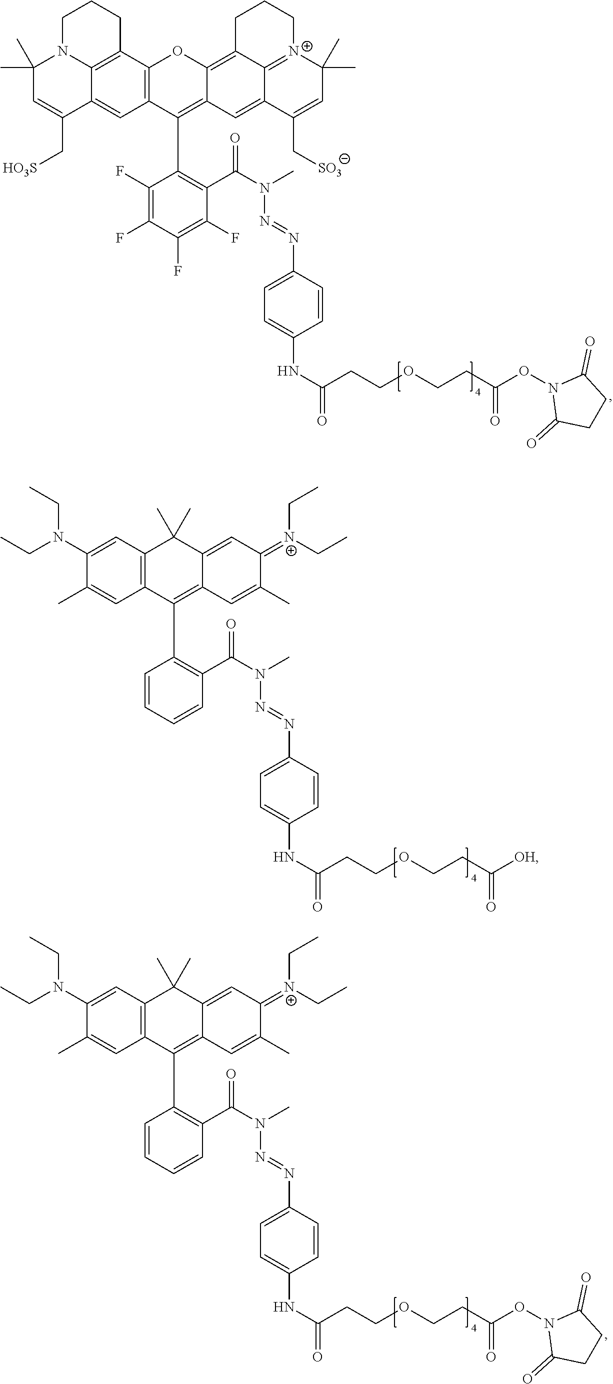

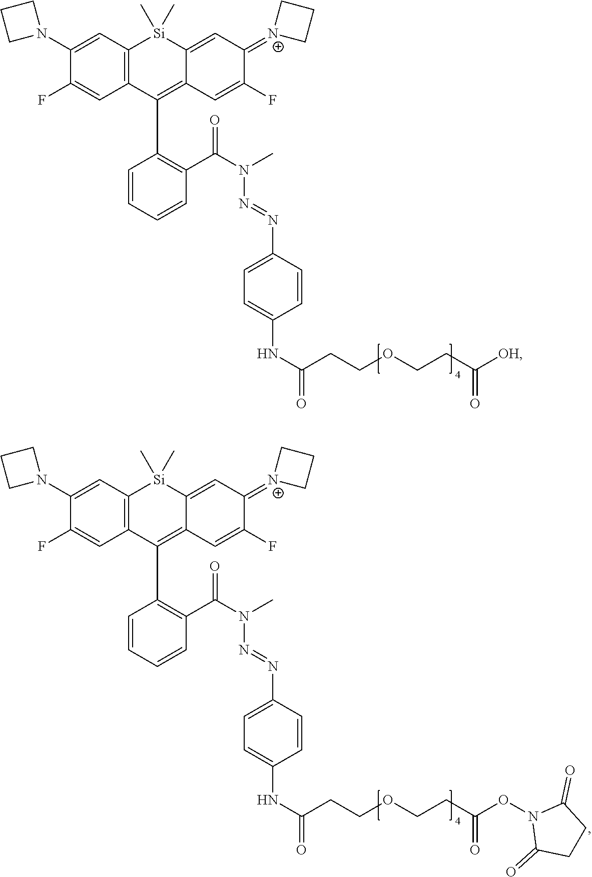

or a pharmaceutically acceptable salt thereof.

In some embodiments, a skilled chemist would be able to select and implement any of the amine protecting groups, alcohol protecting groups, or carboxylic acid protecting groups of the present disclosure. The chemistry of protecting groups can be found, for example, in P. G. M. Wuts and T. W. Greene, Protective Groups in Organic Synthesis, 4^{th} Ed., Wiley & Sons, Inc., New York (2006) (which is incorporated herein by reference), including suitable examples of the protecting groups, and methods for protection and deprotection, and the selection of appropriate protecting groups.

Suitable examples of amine-protecting groups include Carbobenzyloxy (Cbz) group, p-Methoxybenzyl carbonyl (Moz or MeOZ), tert-Butyloxycarbonyl (BOC) group, 9-Fluorenylmethyloxycarbonyl (Fmoc), Acetyl (Ac), Benzoyl (Bz), Benzyl (Bn) group, Carbamate group, p-Methoxybenzyl (PMB), 3,4-Dimethoxybenzyl (DMPM), p-Methoxyphenyl (PMP) group, Tosyl (Ts) group, Troc (trichloroethyl chloroformate), and nosyl group.

Suitable examples of alcohol-protecting groups include acetyl (Ac), benzoyl (Bz), benzyl (Bn), β-methoxyethoxymethyl ether (MEM), dmethoxytrityl, [bis-(4-methoxyphenyl)phenylmethyl](DMT), methoxymethyl ether (MOM), methoxytrityl [(4-methoxyphenyl)diphenylmethyl](MMT), p-methoxybenzyl ether (PMB), methylthiomethyl ether, pivaloyl (Piv), tetrahydropyranyl (THP), tetrahydrofuran (THF), trityl (triphenylmethyl, Tr), silyl ether (most popular ones include trimethylsilyl (TMS), tert-butyldimethylsilyl (TBDMS), tri-iso-propylsilyloxymethyl (TOM), and triisopropylsilyl (TIPS) ethers), methyl ethers, and ethoxyethyl ethers (EE).

Suitable examples of carboxylic acid protecting groups include methyl esters, benzyl esters, tert-butyl esters, esters of 2,6-disubstituted phenols (e.g., 2,6-dimethylphenol, 2,6-diisopropylphenol, 2,6-di-tert-butylphenol), silyl esters, orthoesters, and oxazoline.

Suitable examples of groups reactive with a side chain of an amino acid of a protein are described, for example, in D. Shannon, Covalent protein modification: the current landscape of residue-specific electrophiles, Current Opinion in Chemical Biology 2015, 24, 18-26, which is incorporated herein by reference in its entirety.

Suitable examples of groups reactive with OH of a serine include the following groups:

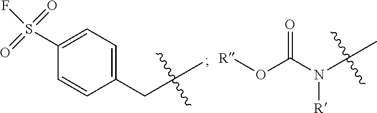

(R′ is H or C₁₋₃ alkyl, R″ is C₁₋₃ alkyl).

Suitable examples of groups reactive with SH of a cysteine include the following groups:

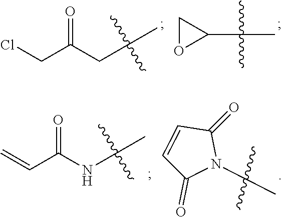

Suitable example of groups reactive with NH₂ of a lysine includes an activated ester of formula:

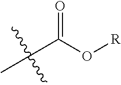

(R is, e.g., N-succinimidyl, N-benzotriazolyl, 4-nitrophenyl, or pentafluorophenyl).

In some embodiments, the compound of Formula (I) is a fluorophore. For example, the compound can by excited by a light of a wavelength form about 300 nm to about 800 nm, and has an emission wavelength from about 500 nm to about 650 nm, from about 550 nm to about 650 nm, or from about 500 nm to about 600 nm. For example, the compounds has emits violet, blue, cyan, green, yellow, orange or red light, which can be detected by fluorescent imaging devices, including the ability to measure the intensity of the fluorescence.

In some embodiments, a salt (e.g., pharmaceutically acceptable salt) of a any compound disclosed herein, including any compound disclosed herein, such as the compound of Formula (I) or Formula (II), is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Protein Conjugates

In some embodiments, the reagents of Formula (I) can be reacted with a protein to obtain a protein conjugate of Formula (II):

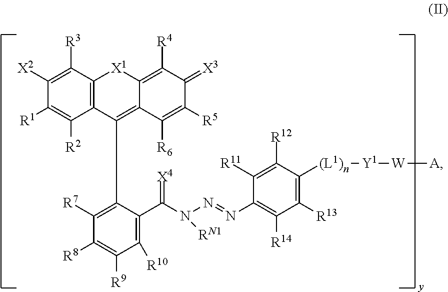

or a pharmaceutically acceptable salt thereof, wherein:
A is a protein;
y is an integer from 1 to 10;
Y¹ is a residue of a group which, prior to conjugation with the protein A, was a group reactive with a side chain of an amino acid of the protein A;
each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A;
X¹ is selected from O, S, C(R¹⁵)₂, and Si(R¹⁵)₂;
each R¹⁵ is independently selected from H, C₁₋₆ alkyl, C₂₋₆ alkenylene, and C₁₋₆ haloalkyl; wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
or any two R¹⁵ together with the C or Si atom to which they are attached from a 3-7 membered saturated or unsaturated carbocyclic or heterocyclic ring, which is optionally substituted with 1 or 2 substituents independently selected from halo OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy;
R^{N1} is selected from C₁₋₃ alkyl and C₁₋₃ haloalkyl, wherein said C₁₋₃ alkyl is optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
X² is selected from OR^{N2} and N(R^{N2})₂;
X³ is selected from 0 and NR^{N2};
X⁴ is 0, S; or NR^{N2};
each R^{N2} is independently selected from H, C₁₋₆ alkyl, and C₁₋₆ haloalkyl, wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
or any two R^{N2} together with the O or N atom to which they are attached from a 3-7 membered saturated or unsaturated carbocyclic or heterocyclic ring, which is optionally substituted with 1 or 2 substituents independently selected from halo, OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy;
R¹, R², R³, R⁴, R⁵, R⁶, R⁷, R⁸, R⁹, R¹⁰, R¹¹, R¹², R¹³, and R¹⁴ are each independently selected from H, halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C₁₋₆ alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃ haloalkoxy;
or each pair of R^{N2} and R³, R^{N2} and R¹, R^{N2} and R⁴, R^{N2} and R⁵, R¹ and R², and R⁵ and R⁶, together with the C, N, or O atoms to which they are attached, form a 5-8 membered saturated or unsaturated carbocyclic or heterocyclic ring, which is optionally substituted with 1, 2, or 3 substituents independently selected from halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C₁₋₆ alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃ haloalkoxy;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each R^{N} is independently selected from H, C₁₋₃ alkyl, and C₁₋₃ haloalkyl; and
each x is independently an integer from 1 to 2,000.

In some embodiments, the protein A is selected from an antibody, an antibody fragment, an engineered antibody, a peptide, and an aptamer.

In some embodiments, the antibody is specific to an antigen which is a biomarker of a disease or condition.

In some embodiments, the disease or condition is cancer.

In some embodiments, y is an integer from 1 to 7. In some embodiments, y is an integer from 1 to 5. In some embodiments, y is selected from 1, 2, 3, 4, 5, 6, or 7. In some embodiments, y is 1.

In some embodiments, the conjugate of Formula (II) has formula:

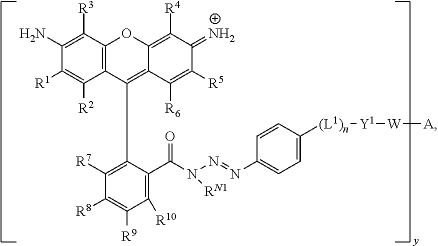

or a pharmaceutically acceptable salt thereof.

In some embodiments:
y is an integer from 4 to 6;
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino,
R⁷, R⁸, R⁹, and R¹⁰ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino; and
R^{N1} is selected from C₁₋₃ alkyl and C₁₋₃ haloalkyl.

In some embodiments:
R¹, R², R³, R⁴, R⁵, and R⁶ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy, wherein said C₁₋₃ alkyl is optionally substituted with OH, SO₃H, C(═O)OH, CN, C₁₋₃ alkoxy, and C₁₋₃ haloalkoxy;
R⁷, R⁸, R⁹, and R¹⁰ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy; and
R^{N1} is C₁₋₃ alkyl.

In some embodiments, the conjugate of Formula (II) has formula:

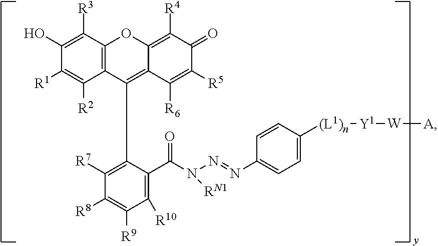

or a pharmaceutically acceptable salt thereof.

In some embodiments:
y is an integer from 4 to 6;
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino,
R⁷, R⁸, R⁹, and R¹⁰ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino; and
R^{N1} is selected from C₁₋₃ alkyl and C₁₋₃ haloalkyl.

In some embodiments:
R¹, R², R³, R⁴, R⁵, and R⁶ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy, wherein said C₁₋₃ alkyl is optionally substituted with OH, SO₃H, C(═O)OH, CN, C₁₋₃ alkoxy, and C₁₋₃ haloalkoxy;
R⁷, R⁸, R⁹, and R¹⁰ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy; and
R^{N1} IS C₁₋₃ alkyl.

In some embodiments, n is an integer from 1 to 5, and each L¹ is selected from NH, O, C(═O), and C₁₋₆ alkylene.

In some embodiments, (L′)ₙ comprises a side chain of an amino acid (e.g., lysine, serine, threonine, cysteine, tyrosine, aspartic acid, or glutamic acid), and Y¹ comprises the terminal functional group of the side chain of the amino acid (e.g., N, O, S, or C(═O)).

In some embodiments, Y¹, prior to conjugation to protein A, is any one of the reactive Y¹ groups described herein for Formula (I). Suitable examples of Y¹ groups of Formula (II) include C(═O), and any one of the following moieties:

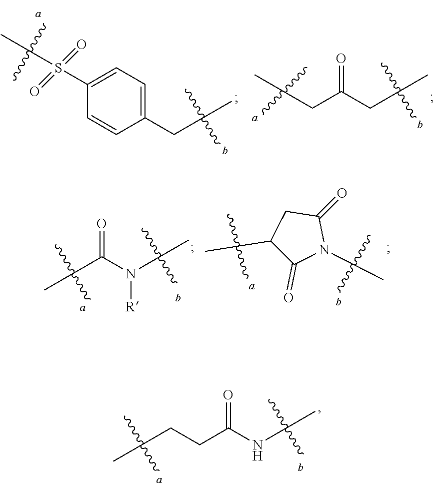

wherein a is point of attachment of the moiety to W, and b is a point of attachment of the moiety to L¹.

In some embodiments, W is O of a side chain of serine, threonine, or tyrosine of the protein A. In some embodiments, W is S of a side chain of cysteine of the protein A.

In some embodiments, W is NH of a side chain of lysine of the protein A. In some embodiments, W is C(═O) of a side chain of aspartic acid or glutamic acid of the protein A.

In some embodiments, each Y¹ is C(═O) and each W is NH of a side chain of lysine of the protein A. In some embodiments, each Y¹ is C(═O) and at least one W is S of a side chain of cysteine of the protein A.

In some embodiments, the protein is selected from an antibody, an antibody fragment, an engineered antibody, a peptide, and an aptamer. In some embodiments, the protein is an antibody. In some embodiments, the antibody is specific to an antigen which is a biomarker of a disease or condition. In some embodiments, the disease or condition is cancer. In some embodiments, the disease or conditions is a disease of the immune system. Suitable examples of such diseases include severe combined immunodeficiency (SCID), autoimmune disorder, familial Mediterranean fever and Crohn's disease (inflammatory bowel disease), arthritis (including rheumatoid arthritis), Hashimoto's thyroiditis, diabetes mellitus type 1, systemic lupus erythematosus, and myasthenia gravis. In some embodiments, the antigen is a biomarker of immune system response to a viral infection or a vaccine. Suitable example of viral infections include infections caused by a DNA virus, an RNA virus, or a coronavirus. One example of a viral infection is influenza. Another example of a viral infection is a coronavirus infection, such as COVID-19 (caused by SARS-CoV-2), Middle East respiratory syndrome (MERS) (caused by MERS-CoV), or severe acute respiratory syndrome (SARS) (caused by SARS-CoV). In some embodiments, the antigen is a biomarker of a cytokine storm. A cytokine storm can occur as a result of an infection (e.g., a viral infection as described herein), a vaccine (e.g., a vaccine against any of the viral infections described herein), an autoimmune condition, or other disease. Suitable examples of such cytokines include pro-inflammatory cytokines such as IL-6, IL-1, TNF-α, or interferon. In some embodiments, the antibody is specific to an antigen indicative of an immune system response to COVID-19 (including cytokine storm).

Suitable examples of biomarkers include CD45, CD3, CD4, CD8, PD-1, PD-L1, CD11b, F4/80, CD163, CD206, Ly6G, CD11c, and MHCII. Any other biomarker the presence of which in the cell (e.g., on the cell surface) is known in the art to be indicative of severity of the disease, or to be indicative of the presence of some disease state, can be used as an antigen for the antibody A of the Formula (II). Some examples of cancer biomarkers include alpha fetoprotein (AFP), CAi5-3, CA27-29, CA19-9, CA-125, calcitonin, calretinin, carcinoembryonic antigen, CD34, CD99MIC 2, CD117, chromogranin, chromosomes 3, 7, 17, and 9p21, cytokeratin (various types: TPA, TPS, Cyfra2l-1), desmin, epithelial membrane antigen (EMA), factor VIII, CD31 FL1, glial fibrillary acidic protein (GFAP), gross cystic disease fluid protein (GCDFP-15), HMB-45, human chorionic gonadotropin (hCG), immunoglobulin, inhibin, keratin (various types), lymphocyte marker (various types, MART-1 (Melan-A), myo D1, muscle-specific actin (MSA), neurofilament, neuron-specific enolase (NSE), placental alkaline phosphatase (PLAP), prostate-specific antigen (PSA), PTPRC (CD45), S100 protein, smooth muscle actin (SMA), synaptophysin, thymidine kinase, thyroglobulin (Tg), thyroid transcription factor-1 (TTF-1), tumor M2-PK, and vimentin.

In some embodiments, the biomarker is selected from CD45, CD3, CD8, CD4, FoxP3, NK1.1, CD19, CD20, CD11b, F4/80, CD11c, Ly6G, Ly6C, MHCII, PD-1, PD-L1, granzyme B, IFNγ, CK5/6, p16, CD56, CD68, CD14, CD1a, CD66b, CD39, TCF1, IL-12β, and CD163. In some embodiments, the antibody is specific to PD-1 (e.g., pembrolizumab, nivolumab, or cemiplimab). In some embodiments, the antibody is specific to PD-L1 (e.g., atezolizumab, avelumab, or durvalumab).

In some embodiments, the present disclosure provides a composition comprising a protein conjugate of Formula (II), or a pharmaceutically acceptable salt thereof, and an inert carrier. In some embodiments, the composition is an aqueous solution (i.e., the inert carrier is water). The aqueous solution may be a buffer, such as any buffer containing inert carrier such as water, phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, or any combination thereof. Some examples of buffers include Dulbecco's phosphate-buffered saline (DPBS), phosphate buffered saline, and Krebs-Henseleit Buffer. The pH of the buffer may be from about 5 to about 9, for example pH may be 6-8. The compound of Formula (I), or a salt thereof, wherein Y¹ is a group reactive with a protein, may be admixed with the protein (e.g., antibody) in any of the aqueous solutions described here to obtain the compound of Formula (II).

A composition (e.g., an aqueous solution) comprising the compound Formula (II), may be used to treat a cell (e.g., a cell containing a biomarker) to image the cell using the fluorophore of the Formula (II).

### Methods of Cellular Analysis

Accordingly, the present disclosure provides a method of examining a cell or a component of a cell (e.g., nucleus of a cell), the method comprising:
(i) contacting the cell with a conjugate of Formula (II) (e.g., the compound having fluorescent properties as described herein), or a pharmaceutically acceptable salt thereof, or a pharmaceutical composition comprising same;
(ii) imaging the cell with an imaging technique; and
(iii) after (ii), contacting the cell with a light of a wavelength.

Without being bound by a theory, it is believed that when the cell is contacted with the protein conjugate of Formula (II) in step (i), the protein A (e.g., antibody such as a cancer biomarker antibody) binds to its antigen on the surface of the cell or in the cytoplasm of the cell (or in a nucleus of the cell), and, therefore, the cell or its component can be imaged by detecting fluorescence of the fluorophore in the Formula (II).

In some embodiments, the imaging technique of step (ii) is a fluorescence imaging, such as microscopy, imaging probes, and spectroscopy. The fluorescence imaging devices include an excitation source, the emitted light collection source, optionally optical filters, and a means for visualization (e.g., a digital camera for taking fluorescence imaging photographs). Suitable examples of fluorescence imaging include internal reflection fluorescence microscopy, light sheet fluorescence microscopy, and fluorescence-lifetime imaging microscopy. Suitable imaging techniques are described, for example, in Rao, J. et al., Fluorescence imaging in vivo: recent advances, Current Opinion in Biotechnology, 18, (1), 2007, 17-25, which is incorporated herein by reference in its entirety.

### Methods of Use

In some embodiments, the present disclosure provides a method of profiling a cell, the method comprising (i) obtaining the cell from a subject, and (ii) examining the cell according to the methods of cellular analysis described herein.

In some embodiments, the present disclosure provides a method of examining a cell using a cytometry technique, the method comprising (i) obtaining the cell from a subject, and (ii) examining the cell according to the method of cellular analysis described herein. Suitable examples of cytometry techniques include image cytometry, holographic cytometry, Fourier ptychography cytometry, and fluorescence cytometry.

In some embodiments, the present disclosure provides a method of diagnosing a disease or condition of a subject by examining pathology of a cell obtained from the subject, the method comprising (i) obtaining the cell from a subject, and (ii) examining the cell according to the method of cellular analysis described herein.

In some embodiments, the present disclosure provides a method of monitoring progression of disease or condition (or monitoring efficacy of treatment of disease or condition) of a subject by examining pathology of a cell obtained from the subject, the method comprising (i) obtaining the cell from the subject, and (ii) examining the cell according to the method of cellular analysis described herein. The method allows to guide therapeutic regimens based on the results of examination of the cell according to the methods, and to provide individualized treatments.

In some embodiments, the present disclosure provides a method of monitoring efficacy of treatment of cancer. Suitable examples of cancer treatments include chemotherapy, radiation therapy, and surgery, or any combination of the foregoing. Suitable examples of chemotherapeutic treatments include abarelix, aldesleukin, alitretinoin, allopurinol, altretamine, anastrozole, arsenic trioxide, asparaginase, azacitidine, bexarotene, bleomycin, bortezomib, busulfan, calusterone, capecitabine, carboplatin, carmustine, chlorambucil, cisplatin, cladribine, clofarabine, cyclophosphamide, cytarabine, dacarbazine, dactinomycin, dalteparin, dasatinib, daunorubicin, decitabine, denileukin, dexrazoxane, docetaxel, doxorubicin, dromostanolone propionate, emtansine, epirubicin, eribulin, erlotinib, estramustine, etoposide, everolimus, exemestane, fentanyl citrate, filgrastim, floxuridine, fludarabine, fluorouracil, fruquintinib, fulvestrant, gefitinib, gemcitabine, ozogamicin, goserelin acetate, histrelin acetate, tiuxetan, idarubicin, ifosfamide, imatinib, interferon α2a, irinotecan, ixabepilone, lapatinib, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, oxaliplatin, paclitaxel, pamidronate, panitumumab, pegaspargase, pegfilgrastim, pemetrexed, pentostatin, pipobroman, plicamycin, procarbazine, quinacrine, rasburicase, sorafenib, streptozocin, sulfatinib, sunitinib, sunitinib, tamoxifen, temozolomide, teniposide, testolactone, thalidomide, thioguanine, thiotepa, topotecan, toremifene, tretinoin, uracil mustard, valrubicin, vinblastine, vincristine, vinorelbine, volitinib, vorinostat, and zoledronate, or a pharmaceutically acceptable salt thereof.

In some embodiments, cancer treatment comprises administering to a patient an antibody useful in treating cancer. Suitable examples of such antibodies include pembrolizumab, nivolumab, cemiplimab, atezolizumab, avelumab, durvalumab, abagovomab, adecatumumab, afutuzumab, alacizumab pegol, altumomab pentetate, amatuximab, anatumomab mafenatox, apolizumab, arcitumomab, bavituximab, bectumomab, belimumab, bevacizumab, bivatuzumab mertansine, blinatumomab, brentuximab vedotin, cantuzumab mertansine, cantuzumab ravtansine, capromab pendetide, cetuximab, citatuzumab bogatox, cixutumumab, clivatuzumab tetraxetan, dacetuzumab, demcizumab, detumomab, drozitumab, ecromeximab, eculizumab, elotuzumab, ensituximab, epratuzumab, etaracizumab, farletuzumab, figitumumab, flanvotumab, galiximab, gemtuzumab ozogamicin, girentuximab, ibritumomab tiuxetan, imgatuzumab, ipilimumab, labetuzumab, lexatumumab, lorvotuzumab mertansine, nimotuzumab, ofatumumab, oregovomab, panitumumab, pemtumomab, pertuzumab, tacatuzumab tetraxetan, tositumomab, trastuzumab, totumumab, rituximab, alemtuzumab, durvalumab, ofatumumab, elotuzumab, and zalutumumab.

Suitable examples of cancer treatments also include immunotherapy. In some embodiments, the cancer treatment comprises a checkpoint inhibitor. In some embodiments, the checkpoint inhibitor is selected from anti-PD-1, anti-PD-L1, anti-CTLA-4, anti-CD20, anti-SLAMF7, and anti-CD52 (e.g., any one of the anticancer antibodies described above).

In some embodiments, the present disclosure provides a method of detecting a disease biomarker in a cell, the method comprising (i) obtaining the cell from a subject, and (ii) examining the cell according to the method of cellular analysis described herein.

In some embodiments, the cell is obtained from the subject using image-guided biopsy, fine needle aspiration (FNA), surgical tissue harvesting, punch biopsy, liquid biopsy, brushing, swab, touch-prep, fluid aspiration or blood analysis. In some embodiments, the cell is obtained from the subject using fine needle aspiration (FNA). In some embodiments, the cell is obtained from a tissue sample, such as a paraffin embedded (FFPE) tissue sample, a fresh tissue sample, or a frozen tissue sample. In some embodiments, the cell is selected from a cancer cell, an immune system cell, and a host cell (the methods of the present disclosure are useful for hepatocyte profiling in liver disease etc.). In some embodiments, the cell is a cancer cell. In some embodiments, the cancer cell is infected with human papillomavirus (HPV). In some embodiments, the cancer is caused by human papillomavirus (HPV). In some embodiments, a cellular sample obtained from the subject or from a tissue of the subject is scant or abundant. In some embodiments, the methods and reagents of the present disclosure are suitable for cellular samples and tissue samples containing any quantity of cells.

In some embodiments, the disease or condition (which can be diagnosed, monitored, or biomarker of which can be detected using the present methods) is cancer. In some embodiments, the methods disclosed herein allow to determine the composition of the tumor microenvironment. Suitable examples of cancer include lymphoma, breast cancer, skin cancer, head and neck cancer, head and neck squamous cell carcinoma (HNSCC), and oral cancer. Other examples of cancers include colorectal cancer, gastric (gastrointestinal) cancer, leukemia, melanoma, and pancreatic cancer, hepatocellular carcinoma, ovarian cancer, endometrial cancer, fallopian tube cancer, lung cancer, medullary thyroid carcinoma, mesothelioma, sex cord-gonadal stromal tumor, adrenocortical carcinoma, synovial sarcoma, bladder cancer, smooth muscle sarcoma, skeletal muscle sarcoma, endometrial stromal sarcoma, glioma (astrocytoma, ependymoma), rhabdomyosarcoma, small, round, blue cell tumor, neuroendocrine tumor, small-cell carcinoma of the lung, thyroid cancer, esophageal cancer, and stomach cancer. The technology is useful for any cancer detectable and compatible with biopsy by direct visualization, palpation, or image guidance.

In some embodiments, the cell is an immune cell. In some embodiments, the cell is selected from a hematopoietic cell, a T cell, a B cell, a NK cell, a myeloid cell, a macrophage, a dendritic cell, a neutrophil, and a monocyte.

Application of these methods is described more fully as follows.

The linkers, reagents, compounds, and methods of the present disclosure can be used at a point-of-care settings. In developed countries, repeat biopsies of ever-smaller lesions are straining accuracy and throughput, while low- and middle-income countries face extremely limited pathology and imaging resources, large case loads, convoluted and inefficient workflows, and lack of specialists. Advantageously, the compounds and methods described here allow for highly precise analysis of scant cancer samples, particularly those obtained by fine needle aspiration of mass lesions.

Accordingly, in some embodiments, the present disclosure provides an image cytometer that allows for automated cell phenotyping of scant cell samples. Various device applications for the methods and compounds of the present applications are described below.

Cellular cancer diagnostics are essential to clinical decision making: establishing the correct diagnosis, choosing the appropriate treatment, enrolling patients in experimental trials, assessing therapeutic efficacy and/or re-staging disease. Today, cancer specimens are commonly obtained by image-guided biopsy, fine needle aspiration (FNA), surgical tissue harvesting, punch biopsies, brushings, swabs, touch-preps, fluid aspiration or blood analysis (leukemia, lymphoma, liquid biopsies). Some of these methods (core and open surgical biopsies for histopathology) yield abundant tissue for sectioning and staining while others (FNA, brushings, touch-preps for cytopathology) yield scant cellular materials. FNA can often be obtained with minimal intervention using small-gauge needles (20-25 G), have very low complication rates and are generally well tolerated.

Rapid onsite assessment of cellular specimens has become increasingly important to narrowing the time between intervention and initiation of therapy, assuring specimen quality for subsequent diagnoses and minimizing sample degradation and loss during transport. The current workflows are still labor intensive and often centralized, requiring extensive sample processing and expert cytopathology review Digital cytopathology and whole-slide imaging have been implemented but also require significant time, labor and investment. Taken together, these factors limit throughput, cost and global reach. A particular challenge is reliably analyzing scant cells either via manual imaging (requiring a trained cytopathologist reviewing an entire slide) or automated analysis (incorporating machine learning routines for automated diagnoses).

The present compounds and methods can be used in automated molecular image cytometers that use advanced materials, engineering and artificial intelligence (AI) for digital cell phenotyping. These new “all-in-one” systems address a potentially large clinical need by enabling advanced cellular diagnostics well suited to: 1) a global health market that is currently underserved; 2) repeat sampling at ultra-low morbidity since smaller needles are used (important for repeat sampling in clinical trials); 3) faster turn-around times (time saved by point-of-care analysis and neither embedding nor staining cores); 4) better and automated quality control and 5) invoking automation to reduce both time to diagnosis and the variability of interpretation. In addition, the present compounds and methods can be used in low-cost flow cytometers, liquid biopsies focusing on cfDNA, exosomes, circulating tumor cells (CTCs), and genomic screening tools (F1CDx, MSK-IMPACT). In some embodiments, the present compounds and methods are useful in automated analysis of cellular specimens obtained by tumor FNA. In some embodiments, the present disclosure provides, in addition to the miniaturized and automated cytometry systems for desktop, point-of-care application described here, a high-throughput device useful for analysis of samples in centralized laboratories, such as CLIA labs.

### Generic Cellular Stains

Conventional cytopathology largely relies on chromogenic stains such as hematoxylin and eosin (H&E), Papanicolaou (PAP) and Giemsa. Stained specimens are reviewed by cytopatholgists who evaluate cells for a number of parameters, for example, nuclear/cytoplamic ratio, nuclear features, mitoses, clusters, cell uniformity, and cohesiveness. Such analyses can be automated but are inherently limited, resulting in variable diagnostic accuracies and lack of molecular information. Most commercial cell analyzers use this approach for automated white blood cell (WBC) analysis rather than cancer detection. Alternative dyes to investigate nuclear morphology (aneuploidy, segmentation) include DAPI, acridine orange, ethidium iodide, propidium iodide or flavins. Given the limitations of generic chromogenic staining, immunostaining for cancer-associated and host cell markers has emerged as an alternative and is being used widely in CTC analysis.

### Antibody Staining

Antibodies are increasingly used in cytopathology and the standard is to perform one stain at a time, primarily using immunocytology (absorption measurements of antibody-enzyme-mediated chemical reactions) rather than immunofluorescence (emission measurements of fluorescently labeled antibodies). The compounds and methods of the present disclosure allow to detect a key molecular biomarker (e.g., cancer biomarker) while allowing morphological assessment of cells (e.g., cancer cells), for example, HER2 immunostaining in H&E slides.

Multichannel fluorescence imaging (typically 4-6 channels) can be used to obtain more stains on a given cell, similar to flow cytometry, albeit at the cost of detailed cellular morphological information. To further improve the number of channels and markers (>20), cycling technologies have been developed that can repeatedly stain, destain and re-stain cancer tissues, ultimately allowing the number of markers per cell to be increased. This in turn facilitates deeper cell-by-cell profiling, pathway analysis and immunoprofiling in scant FNA. Most cycling methods were originally developed for paraffin-embedded tissue sections that can withstand harsh destaining conditions. Unfortunately, these harsh conditions, requiring oxidants for bleaching, are often incompatible with FNA samples. Furthermore, it was not uncommon for early cycling technologies to require days to process samples. Several different cell-compatible cycling technologies have been developed in recent years. The more recent SCANT (single cell analysis for tumor phenotyping) method was shown to be robust and useful for pathway analysis in a clinical setting. One of the obstacles with SCANT, however, was its comparatively low SNR and relatively long destaining times (0.5-1 hour), similar to other cycling techniques. The methods and compounds of the present disclosure (e.g., FAST method) bypasses these shortcomings and allows extremely fast cycling (>95% quenching in <10 sec).

### Choice of Biomarkers

Selecting appropriate molecular markers is essential to identifying cells (e.g., cancer cells), differentiating them from host cells and profiling a growing number of treatment-relevant immune cells. While host cell markers have been thoroughly characterized by extensive flow cytometry studies, epithelial cancer markers are more diverse and thus require more stains. Furthermore, tumor markers are typically only expressed in a fraction of cancer cells and cases. The compounds and methods of the present disclosure allow to stain the following combinations of biomarkers: i) EpCAM, cytokeratins (CK), CD45 and CD16; ii) multi-marker combinations comprising for example EGFR, EpCAM, MUC1 and WNT2 (“Quad“marker”); iii) HER2, ER/PR for breast cancer; iv) CD19/20, k, 1, Ki67 for lymphoma; v) EGFR, TTF1, chromogranin, synaptophysin for lung cancer; vi) EpCAM, calretinin, CD45, vimentin (ATCdx) for ovarian cancer and markers for mutated proteins such as KRASG12d, EGFRv3, IDH1132G and BRAFV600E, among others.

### Optimizing Materials for Cellular Analysis

Freshly obtained clinical samples have to be fixed, stained and captured on glass before they can be analyzed. All of these steps require careful optimization and often modified materials. Fixation can usually be done in paraformaldehyde, methanol/propanol or other commercially available mixes such as CytoRich Red (CRR). We have found empirically that some samples are better preserved in 50% diluted CRR, while fixation length (ideally 15-30 minutes) is less critical.

Immunostaining is best performed in small plastic vials by adding antibody reagents to cells in a staining buffer. Antibody-fluorochrome stability, quality control issues and limited access to basic tools (centrifuge, filters) are notable hurdles when using immunostains in remote areas and in point-of-care (POC) devices. Use of lyophilized antibodies and “cocktails” that contain all necessary ingredients can reduce variability. An alternative is to stain cells directly on glass slides after capture. Capturing cells on a glass slide is also critical to ensure that cells can be brought to the focal plane. Capture can be done using biological “glues” such as dopamine, biotin/neutravidin or polylysine as slide coatings. Alternatively, glass slides can be coated with capture antibodies. Irrespective of the method used, careful validation is required for different applications. Non-specific binding is typically reduced by coating slides with blocking materials such as BSA or PEG polymers. In order to simplify sample handling and processing, commercial systems may adapt cartridges to perform all of the above steps in a single platform.

### Image Cytometry Systems

To inspect heterogeneous cell populations with statistical confidence, image cytometers must visualize large numbers of individual cells. Conventional geometric optics, however, are inherently constrained by the so-called space-bandwidth product (SBP and therefore produce megapixel information. This translates to a familiar experience: common microscopes have either wide field-of-view (FOV) at low resolution or small FOV at high spatial resolution but not both at the same time.

Most laboratory imaging systems overcome this limit by combining high-magnification optics with scanning stages to automatically scan slides and then transmit the information. The technologies of whole side imaging (WSI) and digital cytopathology have progressed over the years but challenges remain. Two key issues in digital cytopathology are i) focusing and ii) the remaining need for expert review. The focusing issue has largely been solved via either autofocusing hardware/software or 3D imaging of thick z-stacks. Autofocusing software often uses either a least squared or a mean value method to locate the ideal focus plane. 3D imaging, such as microscopy with optical sectioning, requires confocal laser scanning microscopy (CLSM), two-photon (2P) microscopy, structured illumination microscopy (SIM), light sheet fluorescence microscopy (LSFM) or Inverted selective plane illumination microscopy (iSPIM). All of these methods entail expensive instrumentation, require expert users and often generate/produce very large data sets. As such, this particular approach limits deployment in resource-constrained remote locations.

New technological advances increasingly enable automated molecular image cytometry, which is particularly helpful for POC use. Computational optics, wherein optically encoded images are digitally interpreted, can expand the SBP beyond optics' physical limit. Advances in optoelectronics and micro-optics further enable the construction of compact, easy-to-control, yet high-performance systems. Using these approaches can also decrease the overall system cost, as optoelectronical parts and computation have become inexpensive. Here we highlight three emerging modalities embodying these new concepts: digital holography, Fourier ptychography and miniaturized fluorescence cytometry.

### Miniaturized Fluorescence Cytometry

As the list of known tumor markers grows, the need for multiplexed cellular profiling also increases, largely driven by interest in improving diagnostic accuracy, allowing patient triaging and facilitating molecularly based treatment decisions. Conventional immunocytology, which is based on chromogenic staining and brightfield microscopy, typically probes only for a few markers simultaneously. Fluorescent imaging, particularly in combination with cycling technologies, is a potent approach to in-depth multiplexing; a major technical challenge is to transform bulky, expensive microscopes into compact, affordable equivalents for POC uses. Fortunately, recent advances in optoelectronics have made available high-quality mini optical 8 parts, prompting new systems engineering. For example, small LEDs can deliver sufficient power to replace conventional lamps or lasers as an excitation light source, and the photosensitivity of semiconductor imagers has improved significantly for reliable low-light detection. Another opportunity is to augment manual image curation with automated analyses using machine learning approaches.

Thumb-sized fluorescent microscopes (“miniscopes”) integrate optical components into a single device. Using a gradient refractive index (GRIN) objective lens makes possible to shorten the optical path and drastically reduce system size (2.4 cm³, 1.9 g). Such a small form factor allowed the scope mountable on an animal's head with minimal interruption to its natural behavior and to image live neuron cells. As potential POC applications, miniscopes have been used for cell profiling and bacterial detection. In addition, a miniscope array performed large-area imaging without scanning, taking advantage of the scope's small lateral size (about 5 mm). System modification and computational processing enabled two-photon excitation, volumetric rendering or lens-less imaging.

For simultaneous multi-color (≥4) cellular analyses, Cytometry Portable Analyzer (CytoPAN) can be used. The system was originally built for operation in remote locations but has additional applications in POC settings (OR, interventional suites, doctors' offices). The excitation light sources were positioned for side illumination through a glass slide, and a single emission filter with four pass bands was used. No dichroic mirrors or mechanical filter changes were necessary. Furthermore, intelligent software streamlined the entire assay, including light-source calibration, sample slide detection, data acquisition and cellular analyses. CytoPAN had four different fluorescent channels and a bright-field imaging capacity. Automated algorithms profiled analyzed individual cells and produced summary reports for cancer diagnosis. This affordable system (<$1,000), in which the compounds and methods of the present application are implemented, is operable by non-skilled workers.

The fluorescent systems discussed above are still bound by the physical SBP limit and there thus remains a trade-off between FOV and spatial resolution. Computational methods used in coherent imaging cannot be applied, because fluorescent emission does not carry phase information. A straightforward workaround is to combine sample scanning with miniaturized optics; a key technical requirement is to automate such operations including stage movement and imaging stitching. Equally important is the development of tools for reliable sample preparation, for example by connecting fluidic cartridges with cost-effective pumping systems. This would speed up assays and minimize procedural errors particularly in cyclic imaging, which requires repeated fluidic handling such as quenching, washing, and labeling.

### Conclusion

In contemporary laboratory medicine, virtually all blood and urine tests have been automated to reduce cost, improve test quality and accommodate the increasing volume of clinical samples. The methods disclosed here allow for automation to be applicable for FNA analysis of cancer samples, particularly in resource-limited environments. Suitable example includes automated POC cytometry, including the rigorous evaluation of cellular markers, staining techniques and kit developments. Automated, AI-based diagnostic DNA-karyometry is another suitable application. Also automated image cytometry, molecularly testing cytology samples, and fluorescent in situ hybridization (FISH) for EGFR, KRAS and BRAF mutation and other cytogenetic abnormalities should be feasible with appropriate amplification strategies. Finally, the compounds and methods of the present disclosure provide the techniques for analyzing FNA specimens for disease (e.g., cancer) diagnosis and monitoring. Inexpensive automated cellular analyses and molecular testing may be contemplated for organ FNA obtained from liver, kidney or blood/bone marrow.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “carboxy” refers to a —C(O)OH group.

As used herein, “halo” refers to F, Cl, Br, or I. In some embodiments, a halo is F, Cl, or Br. The term “perhalo-” (such as “perfluoro-”) refers to groups where each H atom in the group is replaced with a halogen.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C=0), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C=N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C=N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

### EXAMPLES

Materials. All reagents were purchased from commercial sources (Sigma Aldrich, Fischer Scientific, Ambeed, Thermofischer, VWR, see organic synthesis procedures for further details) and used without further purification. Solvents were obtained from Sigma-Aldrich and VWR and deuterated solvents were purchased from Cambridge Isotope Laboratories.

Purification of precursors and intermediates. Precursors were purified using a Biotage SNAP Bio C18 300 Å 4-25 g on a Buchi Pure C-850 FLASHPrep system. Unless stated otherwise, reverse-phase chromatography was performed using water (0.1% formic acid) and acetonitrile (0.1% formic acid) as a gradient (20-40 mL per minute run).

Characterization. NMR spectra were recorded on a Bruker Avance UltraShield 400 MHz spectrometer. ¹H NMR and ¹³C NMR chemical shifts are reported in ppm relative to SiMe₄ (δ=0) and were referenced internally with respect to residual protons (=7.26 for CD₃Cl, δ=7.79 for D₂O, and δ=2.50 for (CD₃)₂SO) and carbons (δ=77.16 for CD₃Cl, and δ=39.52 for (CD₃)₂SO) in the solvent respectively. Coupling constants are reported in Hz. Peak assignments are based on calculated chemical shifts, multiplicity, and 2D experiments. IUPAC names of all compounds are provided and were determined using CS ChemBioDrawUltra 15.

High-performance liquid chromatography-mass spectrometry analysis (HPLC-MS, LCMS) was performed on a Waters instrument equipped with a Waters 2424 ELS Detector, Waters 2998 UV—Vis Diode array Detector, Waters 2475 Multi-wavelength Fluorescence Detector, and a Waters 3100 Mass Detector. Separations employed an HPLC-grade water/acetonitrile solvent gradient with XTerra MS C18 Column, 125 Å, 5 μm, 4.6 mm×50 mm column; Waters XBridge BEH C18 Column, 130 Å, 3.5 μm, 4.6 mm×50 mm. Routine analyses were conducted with 0.1% formic acid added to both solvents.

High-performance liquid chromatography coupled to high-resolution mass spectrometry time of flight (LC-HRMS-ToF). Samples (approximately 100 μmol) were resolved and analyzed using an Agilent 1200 HPLC coupled to an Agilent 6230 TOF. Liquid chromatography was conducted using an IP—RP-HPLC on a 50 mm×2.1 mm (length×i.d.) Xbridge C18 column with 2.5 μm particle size (Waters, Milford, MA) using gradient elution between (A) aqueous 200 mM 1,1,1,3,3,3-hexafluoro-2-propanol with 1 mM triethylamine, pH 7.0, and (B) methanol. Samples were eluted using a gradient of 2.5-95% B over 8 min, at a flow rate of 250 μL/min at 50° C. Samples were analyzed in negative mode from 239 m/z to 3200 m/z. For data collection in positive mode, the column ZORBX 300 SB—C18 (Agilent), 50 mm×2.1 mm (length×i.d.), 1.8 μm particle size was used, with the solvent (A) 0.1% formic acid in water and (B) 0.1% formic acid in acetonitrile. Samples were eluted using a gradient of 10-98% B over 7 min, at a flow rate of 250 μL/min at 50° C. Acquired mass spectra were processed using Agilent's MassHunter software.

Spectroscopy. Stock solutions in dimethyl sulfoxide (DMSO) were prepared at concentrations of 1 mM, stored at −20° C., and thawed immediately before each experiment. Spectroscopic measurements were conducted in phosphate-buffered saline (PBS) pH=7.4 using quartz cuvettes from Thorlabs (10 mm path length). UV-vis measurements were carried out using a Horiba DualFL spectrophotometer (Horiba Instruments). Fluorescence measurements were conducted with a PTI QuantaMaster 400 fluorimeter (Photon Technologies Incorporated, NJ, USA). All measurements were conducted at room temperature. Extinction coefficients were determined by a linear fit of 5 different concentrations for each compound in PBS. Absolute fluorescence quantum yields were determined in PBS (<1 μM) by means of an integrating sphere (Horiba Jobin-Yvon). All spectroscopic experiments were carried out in triplicates.

Photolysis experiments. Solutions for compounds 1a-d were made in the corresponding buffers (pH=3-8) at a concentration of 1-100 μM. The samples were irradiated using different UV light sources (i) LED light source (405 nm, 1.14 mW cm⁻², LuxeonStar SZ-05-U3); UV lamp (˜365 nm, 0.32 mW cm⁻²; UVL-28 EL Series, UVP) at different power and time settings (1 s-5 min). Fluorescence intensity was measured at the corresponding λₘₐₓ of emission before irradiation (t=0 min) and after irradiation (t=1, 2, 3, and 5 min). The data were plotted as the integrated fluorescence intensity normalized to the initial intensity (I=1).

Antibody modification. All commercially available antibodies (FIG. 6) were validated by Western blots and immunofluorescence using non-FLASH-off probes. BSA-free antibodies were exchanged into bicarbonate buffer (pH 8.4) using a 40k zeba column (Thermo Fisher). After buffer exchange, antibodies were incubated with a 5- to 12-fold molar excess of the activated FLASH-off NHS probes for 25 mins at room temperature. The conjugation reaction was loaded onto another 40k zeba column (equilibrated with PBS) for desalting and removal of unreacted dye molecules. The absorbance spectrum of the conjugated antibody was measured using a Nanodrop 1000 (Thermo Scientific) to determine the degree of labeling (DOL), estimating the extinction coefficient from a standard curve of different concentrations, IgG antibody, and correction factor (CF280) for the dye absorbance at 280 nm. The FLASH-off conjugated antibodies were stored in the dark at 4° C. in PBS until usage.

Cell culture. The A431 cell line was used to test and further optimize compounds. Cells were purchased from the American Tissue Culture Collection (ATCC), passaged in DMEM (10% FBS, 1% penicillin/streptomycin) according to the specifications from ATCC. Cells were first grown in a 150 mm cell culture dish and then seeded on Millicell 8-well EZ slides (Millipore) for imaging. After 24-48 hours, confluency wasassessed and cells were fixed with 4% paraformaldehyde in PBS (10 min) prior to EGFR imaging.

Cell immunostaining. Cells were fixed for 10 minutes in 4% PFA and permeabilized for 25 minutes with 0.5% Triton-X100 prior to staining. Immunostaining for FLASH-off imaging was performed in accordance with typical immunofluorescence protocols. After blocking with Intercept Blocking buffer (LI-COR Biosciences) for 30 minutes, cells were stained with FLASH-off conjugated antibodies. Antibodies were diluted to 2-10 μg/ml in Intercept Blocking buffer before staining. Stained cells were washed 3-7 times with PBS before imaging.

Human tissue sections. De-identified formaldehyde-fixed paraffin-embedded tissue sections were commercially obtained from Biomax (HuFOT161, Biomax). Tissue sections were de-paraffinized, rehydrated, and antigen-retrieved in pH 9antigen retrieval buffer. Then sections were blocked with Intercept Blocking buffer (LI-COR) for 30 min before antibody staining for FLASH-off FFPE. Tissues were incubated with FLASH-off antibody conjugates for 30 minutes followed by rinses.

Cytotoxicity Assay. A431 cells were seeded (5000 cells per well) and plated into a 96-well plate (Corning) overnight. Cells were treated with FLASH-off 600 (final concentration=0.1-40 μM) or DMSO as control (<0.5%) in a growth medium. All conditions were incubated for 72 h at 37° C. and 5% CO₂. Cell viability was determined using a Presto Blue assay (Thermofischer) according to the manufacturer's protocol. Statistical analysis was determined from biological triplicates.

Inverted Microscope. An IX81 inverted fluorescence microscope (Olympus, Tokyo, Japan) equipped with a motorized stage (Renishaw, Wotton-under-Edge, England, UK) and fitted with an ORCA-Fusion Digital CMOS camera (Hamamatsu Photonics, Hamamatsu, Japan). Using cellSens Dimension 3.1.1 software (Olympus), multiple fields of view were acquired for each sample with a UPlanSApo ×20 (numerical aperture (NA) 0.75, Olympus) or a UPlanSApo ×40 air objective (NA 0.95, Olympus). In addition to brightfield, four fluorescent channels were acquired. DAPI (345/455), GFP (489/508), YFP (550/565), CY3 (550/565), and CY5 (625/670) were excited with the appropriate optical filters.

Confocal Imaging. Confocal images were collected using a customized Olympus FV1000 confocal microscope (Olympus America). A 2× (XLFluor, NA 0.14), a4× (UPlanSApo, NA 0.16), and an XLUMPlanFL N 20× (NA 1.0) water immersion objective were used for imaging (Olympus America). Probes were excited sequentially using a 405 nm, a 473-nm, a 559-nm, and/or a 633 nm diode laser, respectively, in combination with a DM405/488/559/635-nm dichroic beam splitter. Emitted light was further separated by beam splitters (SDM473, SDM560, and SDM 640) and emission filters BA430-455, BA490-540, BA575-620, and BA655-755 (Olympus America). Confocal laser power settings were carefully optimized to avoid photobleaching, phototoxicity, or damage to the tissue sections. All images were processed using Fiji (ImageJ2, Vers. 2.3/1.53f).

Image and Statistical Analysis. FIJI was used for processing images and GraphPad Prism was used for statistical analysis (Student T-tests, plots). Results were expressed as mean±SEM. Statistical tests included one-way ANOVA followed by Tukey's or Dunnett's multiple comparison test. When applicable, the unpaired one-tailed and two-tailed Student's t tests using Welch's correction for unequal variances were used. Statistical analysis of cell count was done with the spot colocalization plugin (ComDet, Image J). The datasets generated during and/or analyzed during the current study are available from the corresponding author on reasonable request.

### Example 1—Synthesis of Exemplified Compounds

### Synthesis of nitroaryl methyl triazene (E-Z-3-methyl-1-(4-nitrophenyl)triaz-1-ene)

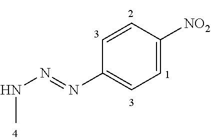

4-nitroaniline (300 mg, 2.17 mmol, Sigma Aldrich) was dissolved in concentrated HCl (3 mL) and diluted water (30 mL). The brown-yellow solution was cooled down to 0° C. using an external ice bath. The aniline was diazotized by drop-wise addition of aqueous sodium nitrite (330 mg, 4.78 mmol, 2.2 eq) in water (20 mL). The disappearance of the starting material was monitored by the loss of color or LCMS. Upon completion, the reaction was allowed to stir for an additional 30 min at 0° C. Aqueous methylamine (40%, 5 mL) was slowly added until the pH of the reaction reached ˜7 (pH paper). The brownish-yellow precipitate was collected by filtration and dried thoroughly under vacuum at 25° C. for 18 h to obtain a yellow solid (320 mg, 78%) consisting of a mixture of isomers.

Note: the desired mono methyl triazene is very reactive and the authors suggest avoiding further purification of the crude material or recrystallization in ethanol or benzene. The crude was characterized by LCMS and was found to be relatively stable when stored dry, under argon at −20° C. for no longer than 14 days.

Health risk: Monomethyl triazenes can be potential carcinogens and careful handling must be taken when handling/synthesizing them.

¹HNMR (400 MHz, (CD₃)₂SO): δ=13.54 (s, 1H, NH), 8.41 (d, J=8.6 Hz, 1H, H2), 8.32 (d, J=8.8 Hz, 1H, H1), 7.94 (d, J=8.6 Hz, 1H, H3), 7.64 (m, 1H, H3), 3.33 (s, overlapping signals, 3H, H4) ppm. MS: molecular weight searched=180.17, m/z found=179.24 [M⁻]. UV-vis: λₘₐₓ 320 nm (1-isomer) and λₘₐₓ 420 nm (2-isomer).

### Synthesis of bis-acyl chloride PEG4 intermediate 6

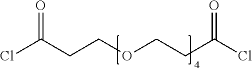

Bis-acid PEG4 (20 mg, 0.07 mmol, BroadPharm) was dissolved in thionyl chloride (100 μL, neat) under inner argon atmosphere. The solution was heated to 50° C. for 1 h. Excess thionyl chloride was removed by the addition of dry toluene (5 mL) and removal of the solvents by rotary evaporation performed in three sequential cycles. The obtained bis-acyl chloride PEG4 (20.1 mg, 90%) 6 was used immediately without further purification or characterization.

### Synthesis of Rhodamine Scaffolds 2a-d .

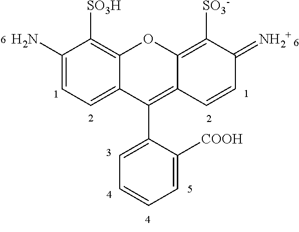

Rhodamine 2a was prepared following previously reported protocols. In brief, rhodamine 110 (250 mg, mmol, Sigma Aldrich) was sulfonated, under vigorous stirring, using fuming sulfuric acid (30% SO₃) at 0° C. for 18 h. The reaction was carefully quenched with ice (30 g) and kept cold (<4° C.). The acidic solution was immediately subjected to reverse-phase chromatography (MeOH:H₂O 1→06%, 0.1% TFA) and the pure fractions were crushed with Et₂O and dried under vacuum for 18 h. The pure bis-sulfonated rhodamine precursor 2a was obtained as an orange solid (113 mg, 38% yield). ¹HNMR (400 MHz, (CD₃)₂SO): δ=8.63 (s, 4H, NH), 8.24 (dd, J=7.8, 1.4 Hz, 1H, H5), 8.02-7.77 (m, 2H, H4), 7.28 (dd, J=7.6, 1.4 Hz, 1H, H3), 7.33-6.93 (m, 4H, H1 & H2) ppm. ¹³CNMR (101 MHz, (CD₃)₂SO): δ=166.35, 155.40, 154.57, 133.14, 131.95, 131.25, 131.22, 130.83, 130.77, 130.66, 130.37, 119.93, 112.98, 110.55 ppm. HRMS (TOF) calculated for [C₂₀H₁₄N₂O₉S₂]: 490.0141 found: 490.0192. UV-vis: λₘₐₓ 494 nm.

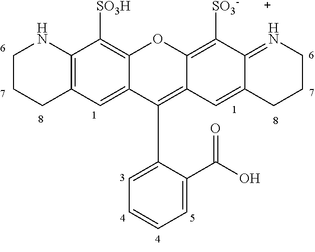

Rhodamine 2b was prepared following previously reported protocols. In brief, Q rhodamine (400 mg, mmol) was sulfonated using fuming sulfuric acid (30% SO₃) at 0° C. for 18 h under vigorous stirring. The reaction was monitored by LCMS and carefully quenched with ice to a final volume of 50 mL of H₂O. The acidic solution was subjected to reverse-phase chromatography (MeOH:H₂O 1→6%, 0.1% TFA) and the pure fractions were crushed with Et₂O and dried under vacuum for 18 h. The pure bis-sulfonated rhodamine precursor 2a was obtained as a dark red solid in 36% yield (200 mg). ¹HNMR (400 MHz, (CD₃)₂SO): δ=9.58 (s, 1H, NH), 8.24 (d, J=7.9 Hz, 1H, H₅), 7.83 (dd, J=15.6, 7.5 Hz, 2H, H₄), 7.43 (d, J=7.4 Hz, 1H, H₃), 6.73 (s, 2H, H₁), 3.55 (m, 4H, H₆), 2.70 (m, 4H, H₈), 1.77 (m, 4H, H₇) ppm. ¹³CNMR (101 MHz, (CD₃)₂SO): δ=166.33, 153.10, 151.30, 133.61, 133.14, 131.36, 130.71, 130.59, 130.50, 128.85, 125.47, 124.09, 112.19, 110.83, 42.20, 27.40, 19.03. ppm. HRMS (TOF) calculated for [C₂₆H₂₂N₂₀₉S₂]: 570.0767 found: 570803. UV-vis: λₘₐₓ 533 nm.

Rhodamine 2c was obtained from a commercial source as the inner salt of rhodamine 101 (Sigma Aldrich, 83694) and used as received without further purification.

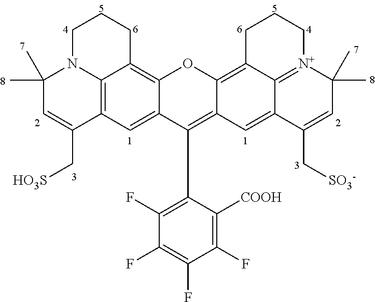

Rhodamine 2d was prepared following previously reported protocols. In brief, the non-sulfonated precursor (300 mg, 0.46 mmol) was sulfonated by the addition of neat sulfuric acid 97% (3 mL) at 0° C. and stirring at 25° C. for 18 h. Completion of the reaction was monitored by LCMS. The reaction was carefully quenched by adding a frozen mixture of 1,4-dioxane (20 mL) and dry Et₂O (50 mL) and stirring for 10 min. Additional Et₂O (400 mL) and hexane (150 mL) were added and allowed to stand at 0° C. for 1 h. The liquid was decanted and the blue viscous oil was further caused with Et₂O (4×100 mL). The remaining residue was dissolved in water and subjected to reverse-phase chromatography (MeCN:H₂O 10-60% with 0.1% FA). The pure fractions were collected, evaporated and the product was dried under high-vacuum for at least 18 h. The product was obtained as a blue solid (146 mg, 39%). ¹HNMR (400 MHz, D₂O): δ=7.09 (s, 2H, H1), 5.90 (s, 2H, H2), 3.93 (d, J=14.5 Hz, 2H, 3H), 3.75 (d, J=14.3 Hz, 2H, 3H), 3.36 (m, 4H, H4), 2.58-2.38 (m, 4H, H5), 1.71 (m, 4H, H6), 1.45 (s, 12H, H7 & H8) ppm. ¹⁹FNMR (377 MHz, D₂O): δ=−136.52 (dt, J=22.2, 5.5 Hz), −137.71 (dt, J=22.2, 5.5 Hz), −150.10 (dt, J=22.2, 5.5 Hz), −154.33 (dt, J=20.9, 5.5 Hz) ppm. HRMS (TOF) calculated for [C₃₈H34F4N₂O₉S₂]: 802.1642 found: 802.1649. UV-vis: λₘₐₓ 628 nm.

Synthesis of Nitro-triazene-rhodamine Intermediates 4a-d

General procedure 1: All coupling reactions with rhodamine precursors 2a-d and nitrotriazene photo-cleavable linker 3 were done following general protocol 1. Under an argon atmosphere and dry conditions, the respective rhodamine (0.1 mmol, 1 eq) was dissolved in anhydrous DMF (2 mL) and treated with DIPEA (0.1 mmol, 1 eq, exclude for 2c) in the presence of about 100 mg molecular sieves (5 Å). The solution was cooled with an external ice bath and solid PyBOP (0.4 mmol, 4 eq) was added in one portion. The reaction was allowed to warm up to room temperature or until full conversion to the reactive HOBT ester (monitored by LCMS, 10 min). The reaction was cooled down with an external ice bath and the solid triazene 3 (30 mg, 0.174 mmol, 1.7 eq,) was added in one portion. Note for compound 2c : dissolve the triazene in 1 mL of DMF and add DIPEA (1 eq) and add the dark purple solution in a moderate drop-wise manner to the rhodamine solution. The solution was allowed to stir for 30 min or until the complete formation of the desired product (monitored by LCMS). The reaction was quenched with water (2 mL) at 0° C. Half the volume of DMF was removed under rotary evaporation (<40° C.) and the remaining solution was subjected to reverse-phase chromatography (MeCN:H₂O 10-60% with 0.1% FA). The pure fractions were collected, evaporated and the product was dried under high-vacuum for at least 18 h.

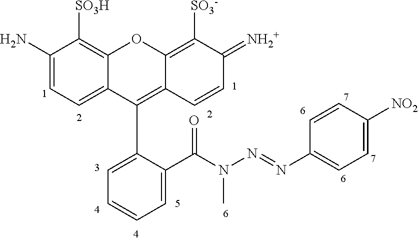

Rhodamine 4a was obtained following general procedure 1. Rhodamine 2a (50 mg, 0.10 mmol), DIPEA (17 μL), PyBOP (211 mg, 0.4 mmol), triazene 3 (30 mg, 0.17 mmol). The product was obtained as an orange solid (53 mg, 80% yield). Note: the product can be recrystallized from methanol. ¹HNMR (400 MHz, (CD₃)₂SO): δ=8.69 (s, 2H, NH), 8.34 (d, J=8.5 Hz, 2H, H7), 7.90 (d, J=7.0 Hz, 1H, H5), 7.83 (d, J=4.9 Hz, 2H, H4), 7.54 (d, J=6.8 Hz, 1H, H3), 7.49 (d, J=8.5 Hz, 2H, H6), 7.01 (d, J=9.3 Hz, 1H, H2), 6.74 (d, J=9.4 Hz, 1H, H1), 3.22 (s, 1H, H8) ppm. ¹³CNMR (101 MHz, (CD₃)₂SO): δ=170.39, 155.94, 154.54, 152.21, 147.21, 134.90, 131.59, 130.79, 130.37, 130.10, 129.86, 129.34, 125.28, 122.72, 119.02, 112.74, 111.58, 28.84 ppm. HRMS (TOF) calculated for [C₂₇H₂₀N₆O₁₀S₂]: 652.0682 found: 652.0713. UV-vis: λₘₐₓ 507 nm.

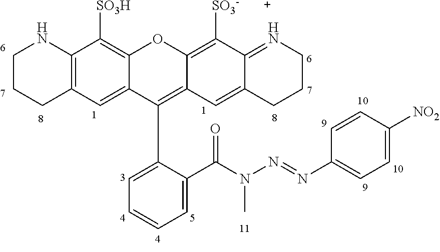

Rhodamine 4b was obtained following general procedure 1. Rhodamine 2b (50 mg, 0.08 mmol), DIPEA (17 μL), PyBOP (182 mg, 0.35 mmol), triazene 3 (30 mg, 0.17 mmol). The product was obtained as an orange solid (45 mg, 70% yield). Note: the product can be recrystallized from methanol. ¹HNMR (400 MHz, (CD₃)₂SO): δ=9.93 (s, 1H, NH), 8.35 (d, J=8.6 Hz, 2H, H10), 8.00 (d, J=7.0 Hz, H5), 7.83 (td, J=5.2, 2.6 Hz, 2H, H4), 7.58 (d, J=8.6 Hz, 2H, H9), 7.53 (d, J=6.2 Hz, 1H, H3), 6.71 (s, 2H, H1), 3.48 (m, 4H, H6), 3.26 (s, 3H, H11), 2.45 (m, 2H, H8) overlapping signals, 2.23 (m, 2H, H8), 1.61 (m, 4H, H7) ppm. ¹³CNMR (101 MHz, (CD₃)₂SO): δ=170.48, 153.25, 152.63, 151.54, 147.43, 135.19, 131.34, 131.10, 130.54, 129.94, 129.62, 128.61, 125.53, 124.77, 123.07, 112.62, 112.17, 41.99, 29.00, 27.34, 19.08. ppm. HRMS (TOF) calculated for [C₃₃H28N₆O₁₀S₂]: 732.1308 found: 732.1319. UV-vis: λₘₐₓ 533 nm.

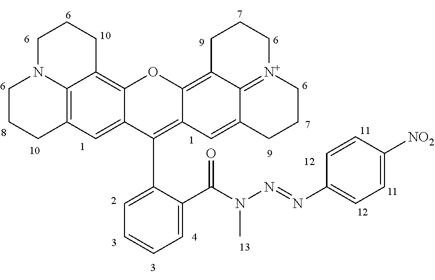 [SMILES: CN(/N=N/c1ccc([N+](=O)[O-])cc1)C(=O)c1ccccc1C1=c2cc3c4c(c2Oc2c1cc1c5c2CCCN5CCC1)CCC[N+]=4CCC3]

Rhodamine 4c was obtained following general procedure 1. Rhodamine 2c (25 mg, 0.08 mmol), PyBOP (105 mg, 0.20 mmol), triazene 3 (10 mg, 0.05 mmol), DIPEA (10 μL). The product was obtained as a purple solid (21 mg, 63% yield). ¹HNMR (400 MHz, (CD₃)₂SO): δ=8.35 (d, J=8.5 Hz, 2H, H12), 8.09-7.87 (m, 1H, H2), 7.82 (m, 2H, H3 & H4) overlapping signals, 7.54 (d, J=8.5 Hz, 2H, H11), 7.48 (m, 1H, H5) overlapping signal, 6.60 (m, 10H, H6) overlapping signal, 3.49 (s, 3H, H13), 3.41-3.20 (m, 4H, H10), 2.41 (m, 2H, H9), 2.19 (m, H2, H9), 1.97 (m, 4H, H8), 1.69 (m, 4H, H7) ppm. ¹³CNMR (101 MHz, (CD₃)₂SO): δ=170.41, 152.39, 151.46, 151.06, 150.52, 147.19, 135.12, 131.26, 130.77, 130.33, 129.65, 129.26, 125.67, 125.30, 123.33, 122.78, 111.95, 104.90, 50.21, 49.74, 28.73, 26.74, 20.03, 19.26, 19.07 ppm. HRMS (TOF) calculated for [C₃₉H37N₆O₄]: 653.2871 found: 653.2892. UV-vis: λₘₐₓ 589 nm.

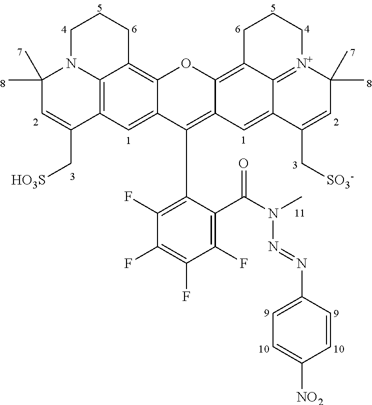

Rhodamine 4d was obtained following general procedure 1. Rhodamine 2d (53 mg, 0.07 mmol), PyBOP (137 mg, 0.26 mmol), triazene 3 (30 mg, 0.17 mmol), DIPEA (17 μL). The product was crushed with tera-butyl methyl ether (TBME), dried and obtained as a blue solid (51 mg, 80% yield). ¹HNMR (400 MHz, CDCl₃): δ=8.43 (s, 1H, OH), 8.30 (d, J=8.9 Hz, 2H, H10), 7.47 (d, J=8.9 Hz, 1H, H9), 6.69 (s, 1H, H1), 6.18 (s, 1H, H1), 5.55 (s, 1H, H2), 5.27 (s, 1H, H2), 3.58 (m, J=24.9 Hz, 4H, H3), 3.39 (s, 3H, H11), 2.99 (m, 4H, H4), 2.10 (m, 4H, H5), 1.97 (m, 4H, H6), 1.55 (s, 3H, H7), 1.53 (s, 3H, H7), 1.48 (s, 3H, H8), 1.41 (s, 3H, H8) ppm. ¹³CNMR (101 MHz, CDCl₃): δ=155.78, 154.88, 154.83, 152.39, 145.80, 138.70, 138.65, 125.23, 124.88, 124.14, 123.67, 123.43, 122.81, 115.88, 115.68, 115.19, 114.84, 107.55, 107.49, 61.53, 55.12, 53.79, 44.79, 28.83, 28.66, 27.52, 21.84, 21.27. HRMS (TOF) calculated for [C₄₅H40F₄N₆O₁₀S₂]: 964.2183 found: 964.2182. UV-vis: λₘₐₓ 637 nm.

### Synthesis of Aniline-Triazene Rhodamine Intermediates 5a-d

General procedure 2: Unless stated otherwise, the hydrogenation of compounds 4a-d was achieved following the general protocol 2. The respective rhodamine (1 eq) was dissolved in methanol (7 mL) in the presence of acetic acid was added. The solution was cooled with an external ice bath and palladium in carbon (10%) was added in one portion. Hydrogen gas was constantly bubbled through the reaction at 0° C. to avoid hydrolysis to the methyl xanthamide. The reaction progressed cleanly via the nitroso adduct and was monitored every 30 mins by LCMS. Upon completion (about 45 min), the reaction was quenched with water (2 mL), filtered through a 0.22 m syringe filter (Amicon), and washed thoroughly with methanol (3 mL) and water (3 mL). The solvents were evaporated by rotary evaporation (<40° C.) keeping the product constantly shielded from light. Because the electron-rich aniline analogs 5a-d were prone to hydrolysis in solution, the obtained intermediates were used without further purification and were characterized by LCMS. The anilines 5a-d were dried under vacuum and used promptly or stored at −20° C. under argon atmosphere. Note: although clean, a minimal amount of palladium is preferred for sulfonated probes. Adsorption of sulfonated probes to the catalyst appears to have a negative effect on yield.

Rhodamine 5a was obtained following general procedure 2. Compound 4a (50 mg, 0.08 mmol), methanol (7 mL), acetic acid (50 μL), Pd/C (10 mg). The product was obtained as an orange solid (15 mg, 31% yield). HRMS (TOF) calculated for [C₂₇H22N₆O₈S2]: 622.0941 found: 622.0942. UV-vis: λₘₐₓ 507 nm.

Rhodamine 5b was obtained following general procedure 2. Compound 4b (20 mg, 0.03 mmol), methanol (4.5 mL), acetic acid (25 μL), Pd/C (5 mg). The product was obtained as a red solid (10 mg, 52% yield). HRMS (TOF) calculated for [C₃₃H30N₆O₈S₂]: 702.1567 found: 702.1583. UV-vis: λₘₐₓ 543 nm.

Rhodamine 5c was obtained following general procedure 2. Compound 4c (25 mg, 0.04 mmol), methanol (7 mL), acetic acid (50 μL), Pd/C (20 mg). The product was obtained as a purple solid (21 mg, 95% yield). HRMS (TOF) calculated for [C₃₉H39N₆O₂]: 623.3129 found: 623.3105. UV-vis: λₘₐₓ 587 nm.

Rhodamine 5d was obtained following general procedure 2. Compound 4d (30 mg, 0.05 mmol), methanol (7 mL), acetic acid (50 μL), Pd/C (10 mg). The product was obtained as a blue solid (20 mg, 45% yield). HRMS (TOF) calculated for [C₄₅H42F₄N₆O₈S2]: 934.2442 found: 934.2435. UV-vis: λₘₐₓ 636 nm.

### Synthesis of FLASH-Off Probes.

General procedure 3: All pegylation reactions to obtain final compounds 1a-d and their NHS analogs (FLASH-off probes) were achieved following general procedure 3. In anhydrous conditions, the rhodamine 5a-d (1 eq) was dissolved in DMF (3 mL) and diluted with DCM (3 mL). The solution was cooled to 0° C. and kept under argon. In a separate vial, the bis-acylchloride PEG46 (3 eq) was dissolved in DCM (3 mL) and cooled to 0° C. Rhodamine was added in a moderate drop-wise manner under constant stirring and constant monitoring by LCMS. For obtaining the acid analogs 1a-d , the reaction was quenched with water (2 mL) and DIPEA (10 μL). For obtaining the respective FLASH-off probes, the reaction was quenched with an anhydrous solution of NHS (2 eq) and DIPEA (1 eq) in DCM (1 mL). The solvents were carefully evaporated and the crude was subjected to reverse-phase chromatography (MeCN:H₂O 5-4% with 0.1% FA). The pure fractions were collected, evaporated and the final products were lyophilized. Note: The sulfonated probes are prone to hydrolysis of the NHS ester. Acid analogs 1a-d were characterized by NMR and spectroscopy. The NHS analogs were characterized by LCMS and stored in dry DMSO aliquots at −20° C. The latter were used for constructing antibody conjugates.

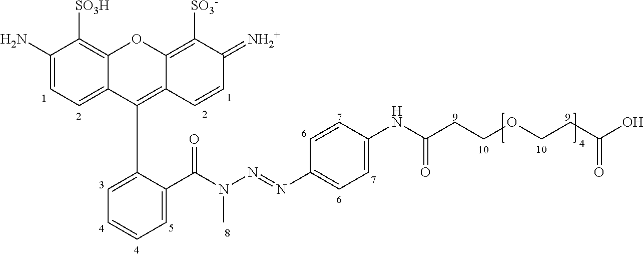

Rhodamine 1a was obtained from compound 5a (10 mg, 0.017 mmol, 1 eq), compound 6 (15.9 mg, 0.05 mmol, 3 eq.), DIPEA (10 μL). The product was obtained as an orange solid (12 mg, 83% yield). ¹HNMR (400 MHz, (CD₃)₂SO): δ=8.70 (s, 4H, NH), 8.33 (d, J=8.5 Hz, 2H, H7), 8.02-7.86 (m, 3H, H5 & H4), 7.83 (d, J=5.3 Hz, 1H, H3), 7.52 (d, J=8.5 Hz, 2H, H6), 7.00 (d, J=9.3 Hz, 2H, H2), 6.73 (d, J=9.4 Hz, 2H, H1), 3.22 (s, 3H, H8), 3.01 (m, 10H, H9), 1.73 (m, 10H, H10) ppm. ¹³CNMR (101 MHz, (CD₃)₂SO): δ=170.39, 155.85, 155.57, 154.53, 152.21, 147.21, 134.91, 131.51, 130.77, 130.41, 130.09, 129.83, 129.31, 126.43, 125.28, 122.71, 119.03, 112.73, 112.39, 45.91, 45.87, 28.83, 25.97, 25.89. ppm. HRMS (TOF) calculated for [C₃₉H42N₆O₁₅S₂]: 898.2150 found: 898.2138. UV-vis: λₘₐₓ 506 nm. FLASH-off 500: Compound 5a (10 mg, 0.017 mmol, 1 eq), compound 6 (15.9 mg, 0.05 mmol, 3 eq), NHS (4 mg, 0.032 mmol), DIPEA (10 μL). The product was obtained as an orange solid (7 mg, 43% yield). HRMS (TOF) calculated for [C₄₃H45N₇O₁₇S₂]: 995.2313 found: 995.2321. UV-vis: λₘₐₓ 506 nm.

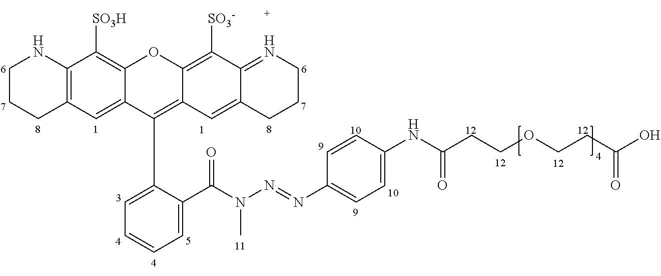

Rhodamine 1b was obtained following general procedure 3. Compound 5b (10 mg, 0.014 mmol, 1 eq), compound 6 (14 mg, 0.042 mmol, 3 eq), DIPEA (10 μL). The product was obtained as an orange solid (4 mg, 28% yield). ¹HNMR (400 MHz, (CD₃)₂SO): δ=10.21 (s, 1H, OH), 7.88-7.78 (m, 3H, H3 & H4), 7.70 (d, J=8.5 Hz, 2H, H10), 7.57-7.43 (m, 1H, H5), 7.35 (d, J=8.5 Hz, 2H, H9), 6.68 (s, 2H, H1), 3.56-3.39 (m, 24H, H9 & H12) overlapping signals, 3.18 (s, 3H, H11), 2.42 (m, 2H, H8) overlapping signals, 2.23 (m, 2H, H8), 1.59 (m, 4H, H7) ppm. ¹³CNMR (101 MHz, (CD₃)₂SO): δ=172.87, 170.22, 169.83, 163.29, 153.65, 151.81, 143.47, 140.71, 135.95, 128.55, 124.25, 122.76, 119.74, 112.29, 69.98, 69.91, 69.89, 69.84, 66.76, 66.44, 41.80, 37.45, 34.95, 28.15, 27.34, 19.23 ppm. HRMS (TOF) calculated for [C₄₅H50N₆O₁₅S₂]: 978.2776 found: 978.2774. UV-vis: λₘₐₓ 542 nm. FLASH-off 550: Compound 5b (4 mg, 0.005 mmol, 1 eq), compound 6 (5.66 mg, 0.017 mmol, 3 eq), NHS (1.3 mg, 0.011 mmol), DIPEA (10 μL). The product was obtained as an orange solid (2 mg, 32% yield). HRMS (TOF) calculated for [C₄₉H53N₇O₁₇S₂]: 1075.2939 found: 1075.2942. UV-vis: λₘₐₓ 543 nm.

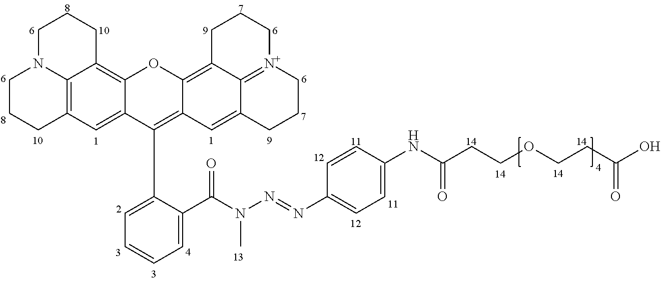

Rhodamine 1c was obtained following general procedure 3. Compound 5c (40 mg, 0.064 mmol, 1 eq), compound 6 (63 mg, 0.19 mmol, 3 eq), DIPEA (30 μL). The product was obtained as an orange solid (22 mg, 388% yield). ¹HNMR (400 MHz, (CD₃)₂SO): δ=10.32 (s, 1H, OH), 7.87-7.73 (m, 3H, H2 & H3) overlapping signals, 7.70 (d, J=8.5 Hz, 2H, H11), 7.46 (dd, J=5.7, 3.2 Hz, 1H, H4), 7.29 (d, J=8.6 Hz, 2H, H12), 6.61 (s, 2H, H1), 3.70 (t, J=6.1 Hz, 2H, H14), 3.57 (t, J=6.4 Hz, 2H, H14), 3.58-3.33 (m, 20H, H6 & H14) overlapping signals, 3.12 (s, 3H, H13), 2.93 (d, J=6.6 Hz, 4H, H10), 2.58 (t, J=6.2 Hz, 2H, H14), 2.38 (m, 4H, H9), 2.26-2.09 (m, 2H, H14), 1.96 (m, 4H, H8), 1.68 (m, 4H, H7) ppm. ¹³CNMR (101 MHz, (CD₃)₂SO): δ=172.77, 170.19, 169.67, 151.75, 151.06, 150.50, 143.16, 140.55, 135.88, 131.00, 130.25, 130.16, 129.37, 129.10, 125.84, 123.26, 122.45, 119.46, 111.95, 104.80, 69.77, 69.70, 69.58, 66.55, 66.47, 54.94, 50.18, 49.71, 37.22, 35.11, 27.92, 26.68, 20.08, 19.26, 19.08 ppm. HRMS (TOF) calculated for [C₅₁H59N₆O₉]: 899.4338 found: 899.4363. UV-vis: λₘₐₓ 588 nm. FLASH-off 600: Compound 5c (10 mg, 0.016 mmol, 1 eq), compound 6 (15.9 mg, 0.05 mmol, 3 eq), NHS (4 mg, 0.032 mmol), DIPEA (10 μL). The product was obtained as an orange solid (8 mg, 50% yield). HRMS (TOF) calculated for [C₅₅H62N_{7011]: 996.4502} found: 996.4528. UV-vis: λₘₐₓ 588 nm.

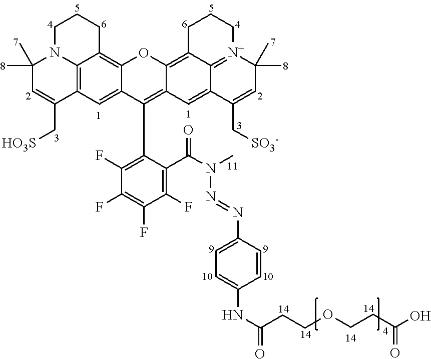

Rhodamine 1d was obtained following general procedure 3. Compound 5d (22 mg, 0.023 mmol, 1 eq), compound 6 (23 mg, 0.07 mmol, 3 eq), DIPEA (10 μL). The product was obtained as an orange solid (6 mg, 21% yield). ¹HNMR (400 MHz, (CD₃)₂SO): δ=7.71 (d, J=8.4 Hz, 2H, H10), 7.47 (s, 1H, H1), 7.41 (d, J=8.4 Hz, 2H, H9), 7.14 (s, 1H, H1), 6.53 (s, 1H, H2, NH), 5.84 (s, 1H, H2), 5.44 (s, 1H, H2), 3.71 (m, 4H, H3), 3.54-3.44 (m, 20H, H14), 3.15 (s, 3H, H11), 2.91 (m, 4H, H6), 2.60-2.43 (m, 4H, H4) overlapping signals, 1.94 (m, H4, H5), 1.51 (s, 3H, H7), 1.47 (s, 3H, H7), 1.38 (s, 3H, H8), 1.32 (s, 3H, H8) ppm. ¹⁹FNMR (377 MHz, (CD₃)₂SO): δ=−133.58 (dt, J=22.2, 5.5 Hz), −137.84 (dt, J=22.2, 5.5 Hz), −150.23 (dt, J=22.2, 5.5 Hz), −154.12 (dt, J=20.9, 5.5 Hz) ppm. ¹³CNMR (101 MHz, (CD₃)₂SO): δ=172.85, 169.78, 163.29, 160.28, 152.39, 150.39, 142.28, 141.21, 141.01, 135.39, 123.14, 121.11, 120.00, 105.56, 69.99, 69.90, 69.84, 66.79, 66.45, 46.12, 46.08, 43.16, 43.09, 37.27, 34.96, 28.69, 28.24, 27.94, 26.18, 26.10, 20.19, 19.51 ppm. HRMS (TOF) calculated for [C₅₇H62F₄N₆O₁₅S₂]: 1210.3651 found: 1210.3602. UV-vis: λₘₐₓ 636 nm. FLASH-off 650: Compound 5d (3 mg, 0.003 mmol, 1 eq), compound 6 (3.18 mg, 0.01 mmol, 3 eq), NHS (4 mg, 0.032 mmol), DIPEA (10 μL). The product was obtained as an orange solid (2 mg, 47% yield). HRMS (TOF) calculated for [C₆₁H65F₄N₇O₁₇S₂]: 1307.3814 found: 1307.383. UV-vis: λₘₐₓ 636 nm.

### Synthesis of Methyl Xanthamides 7a-d.

General procedure 4: All coupling reactions to obtain photoproducts 7a-d were achieved with an adapted version of procedure 1, procedure 4. Rhodamines 2a-d (1 eq, ˜0.2 mmol) were dissolved in dry DMF (1 mL) and treated with DIPEA (0.1 mmol, 1 eq, exclude for 2c). Activation of the carboxylic acid was activated by adding PyBOP (3 eq) in one portion at 0° C. The reaction was allowed to warm up to room temperature or until full conversion to the reactive ester (monitored by LCMS, 10 min). Aqueous methylamine (40%, 3 eq) was added at 25° C. and allowed to stir for 10 min. Upon completion (monitored by LCMS), the reaction was quenched with water (2 mL). Half the volume of DMF was removed under rotary evaporation (<40° C.) and the remaining solution was subjected to reverse-phase chromatography (MeCN:H₂O 10-60% with 0.1% FA). The pure fractions were collected, evaporated and the product was dried under high-vacuum for at least 18 h.

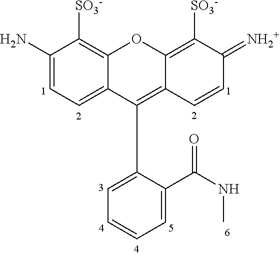

Rhodamine 7a: Rhodamine 2a (10 mg, 0.02 mmol, leq), DIPEA (5 μL), PyBOP (31.7 mg, 0.06 mmol), CH₃NH₂ aq (30 μL), triazene 3 (20 mg, 0.11 mmol). The product was obtained as an orange solid (6 mg, 58% yield). Note: Upon standing in (CD₃)₂SO the compound underwent hydrolysis of one sulfonate ion (423 M⁻). ¹H NMR (400 MHz, (CD₃)₂SO): δ=7.78 (d, J=6.9 Hz, 1H, H5), 7.52 (t, J=6.1 Hz, 2H, H4), 7.05 (d, J=6.8 Hz, 1H, H3), 6.46 (d, J=8.8 Hz, 2H, H2), 6.29 (d, J=8.8 Hz, 2H, H1), 2.50 (s, 3H, H6) overlapping signals, ppm. ¹³CNMR (101 MHz, (CD₃)₂SO): δ=166.88, 153.25, 149.37, 147.72, 133.29, 130.59, 129.75, 129.09, 123.83, 123.00, 114.37, 111.82, 104.51, 63.65, 24.72 ppm. HRMS (TOF) calculated for [C₂₁H17N₃O₈S2]: 503.0457 found: 503.0479. UV-vis: λₘₐₓ 494 nm.

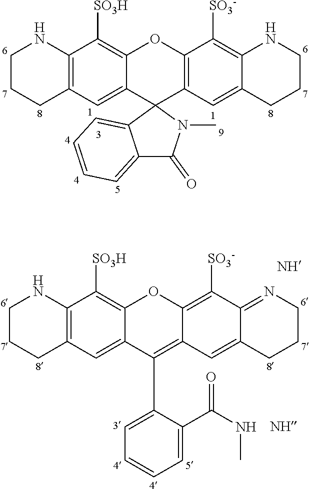

Rhodamine 7b: Rhodamine 2b (28 mg, 0.049 mmol, 1 eq), PyBOP (76 mg, 0.14 mmol), CH₃NH₂ (50 μL) gave the desired product as a pink-white solid (20 mg, 78% yield). Note: the solution in (CD₃)₂SO) gave a mixture of isomers (see LCMS data). ¹HNMR (400 MHz, (CD₃)₂SO): δ=10.12 (s, 1H, NH″), 8.57 (s, 1H, NH), 7.97-7.87 (m, 4H, H4, H4″, H5 and H5″), 7.77-7.33 (m, 2H, H3 and H3″), 7.16-6.99 (m, 2H, H1′), 6.06 (s, 2H, H1), 3.52 (m, 4H, H6) overlapping signal, 3.27 (s, 3H H9), overlapping signal, 2.67 (m, 4H, H8), 1.81-1.49 (m, 8H, H7 and H7″) ppm. ¹³CNMR (101 MHz, (CD₃)₂SO): δ=166.48, 163.09, 152.90, 147.58, 143.57, 132.88, 130.07, 128.60, 128.19, 123.36, 122.61, 118.64, 110.42, 102.67, 63.19, 40.57, 27.19, 24.39, 19.91 ppm. HRMS (TOF) calculated for [C₂₇H25N₃O₈S₂]: 583.1083 found: 583.1102. UV-vis: λₘₐₓ 536 nm.

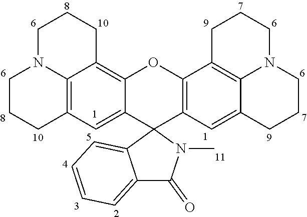 [SMILES: CN1C(=O)c2ccccc2C12c1cc3c4c(c1Oc1c2cc2c5c1CCCN5CCC2)CCCN4CCC3]

Rhodamine 7c: Rhodamine 2c (28 mg, 0.05 mmol, 1 eq), PyBOP (76 mg, 0.14 mmol), CH₃NH₂ (50 μL) gave the desired product as a white solid which was crushed with hexane and crystalized in DMSO (15 mg, 87% yield). ¹HNMR (400 MHz, (CD₃)₂SO): δ=7.71 (dd, J=16.1, 7.3 Hz, 1H, H2), 7.52-7.36 (m, 2H, H4 & H5) overlapping signals, 6.96 (d, J=6.9 Hz, 1H, H5), 5.87 (s, 2H, H1), 3.08 (dt, J=20.8, 5.9 Hz, 4H, H6), 3.08 (dt, J=20.8, 5.9 Hz, 4H, H6), 2.83 (t, J=6.6 Hz, 4H, H10), 2.49 (m, 7H, H9 & H11) overlapping signals, 2.40 (m, 4H, H8), 1.94 (m, 4H, H7) ppm. ¹³CNMR (101 MHz, (CD₃)₂SO): δ=167.05, 147.61, 143.38, 132.49, 131.75, 131.66, 128.72, 123.42, 123.00, 122.39, 117.06, 107.34, 104.99, 67.45, 64.33, 49.17, 48.70, 29.83, 28.41, 23.28, 22.45, 18.60, 13.95, 10.85. ppm. HRMS (TOF) calculated for [C₃₃H34N₃O₂]: 504.2646 found: 504.2666. UV-vis: λₘₐₓ 227 nm (spirocyclic form).

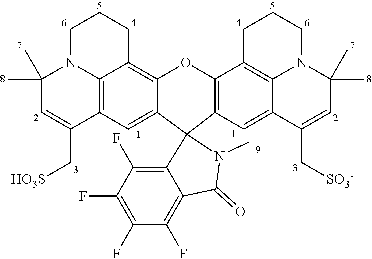

Rhodamine 7d: Rhodamine 2d (10 mg, 0.012 mmol, 1 eq), PyBOP (31.7 mg, 0.06 mmol), CH₃NH₂ (50 μL) gave 6 mg of desired product as blue-white solid (49% yield). ¹HNMR (400 MHz, D₂O): δ=7.17 (s, 2H, H1), 5.77 (s, 2H, H2), 3.87-3.42 (m, 10H, H3, H9 and H4) overlapping signals, 2.94 (m, 4H, H5), 1.97 (s, 4H, H6), 1.46 (s, 12H, H7 & H8) ppm. ¹³CNMR (101 MHz, D₂O): δ=154.66, 154.62, 152.18, 145.59, 138.49, 138.44, 125.02, 124.67, 123.93, 123.22, 122.59, 114.99, 114.62, 107.33, 107.28, 61.32, 54.90, 53.58, 44.57, 28.62, 28.44, 28.32, 21.62, 21.06. HRMS (TOF) calculated for [C₃₉H37F₄N₃O₈S₂]: 815.1958 found: 815.1923. UV-vis: λₘₐₓ 632 nm.

### Example 2—Properties of Pulse-Inactivatable Fluorochromes

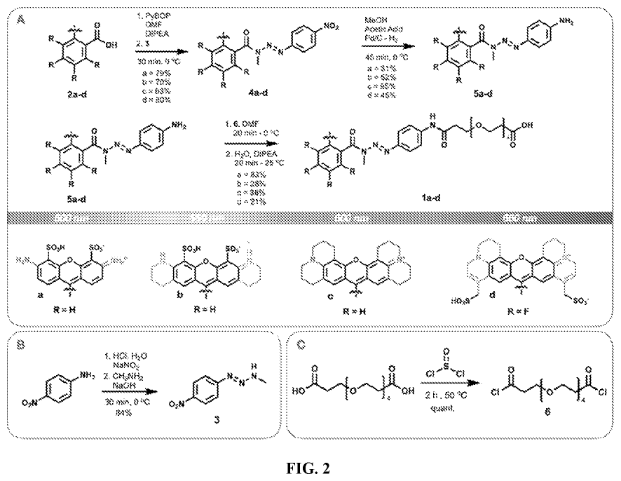

Spectrally distinct, bright, and photo-stable fluorochromes that are rapidly inactivated by a pulse of UV/blue light (350-405 nm) were obtained based on four common rhodamine scaffolds 2a-d with free and substituted anilines and variable length in their conjugation systems and a broad range of emission wavelengths (e.g., about 500-about 650 nm, FIG. 2A). Photo-responsive triazene linkers (e.g., triazene linker 3) were developed that is stable enough to withstand spontaneous hydrolysis during synthesis or imaging and function independently in the presence of reducing agents, in contrast to recently reported reductively-cleavable photosensitized linkers. Once installed, the linker allowed for further chemical modifications to attach handles for conjugating macromolecules to the fluorochromes. After absorbing UV/blue light (about 350-about 405 nm), the linker i) undergoes photolytic cleavage ii) releases a non-cytotoxic photoproduct (e.g., N₂) and iii) diffuses freely from the place of activation as a non-fluorescent adduct.

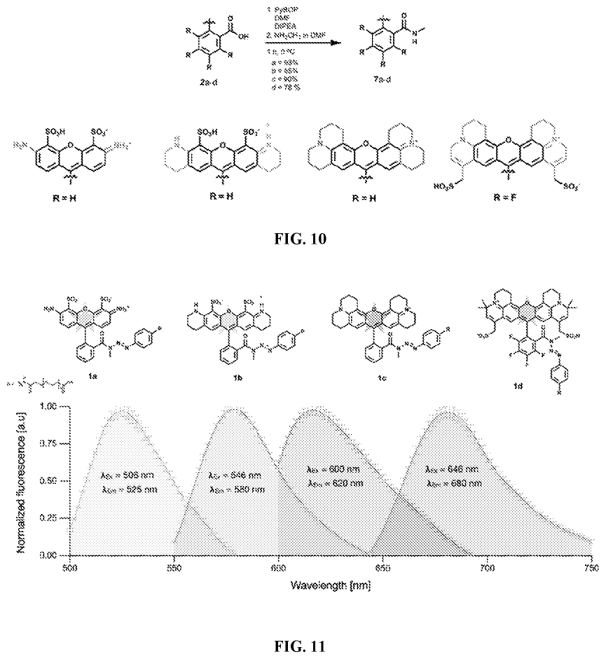

The transformation of rhodamines into FLASH-off probes was achieved by a three-step synthetic route starting from the parent rhodamine scaffolds 2a-d. Triazene 3 was synthesized by diazotization of 4-nitro aniline with NaNO₂ in HCl followed by the addition of methyl amine and NaOH (FIG. 2B). Optimal coupling of triazene 3 to precursors 2a-d was achieved via the HOBt reactive ester. These fast coupling reactions yielded the nitro-containing intermediates 4a-d. These stable analogs were further subjected to Pd-catalyzed hydrogenation with H2, to afford the respective aniline precursors 5a-d. A polyethylene glycol (PEG) spacer was synthesized by treatment of bis-acid PEG4 with thionyl chloride to afford the bis-acyl chloride PEG4 intermediate 6 (FIG. 2C). The PEG linker 6 was installed on the anilines 5a-d to yield the final FLASH-off probes 1a-d. Interestingly, mass spectrometry of rhodamine precursors 5a-d and final compounds 1a-d showed consistent fragmentation to a mass that corresponded with that of the desired methyl xanthamide photoproduct (FIG. 10). The corresponding methyl xanthamides 7a-d were synthesized by direct coupling of precursors 2a-d using methylamine, to compare the retention times and mass-to-charge ratio to the detected photoproducts (see synthesis and FIG. 10).

### Example 3—Photophysical and Photochemical Experiments

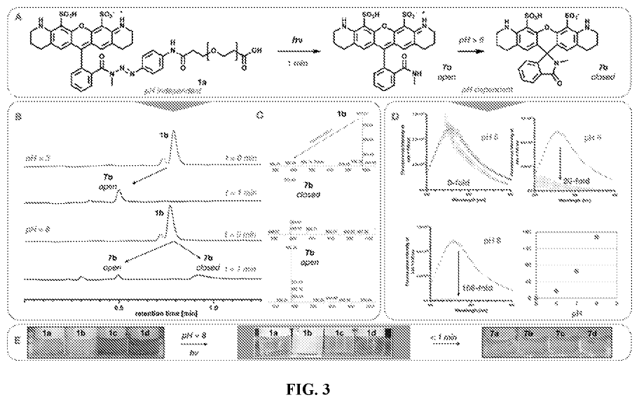

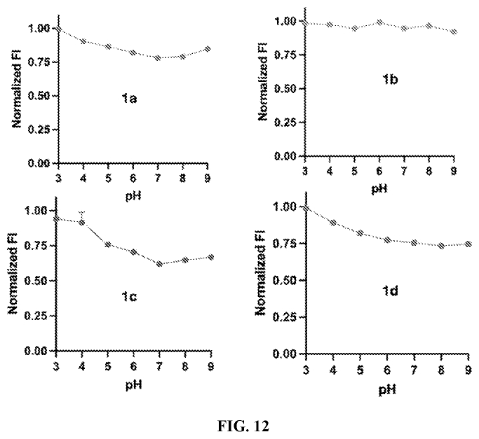

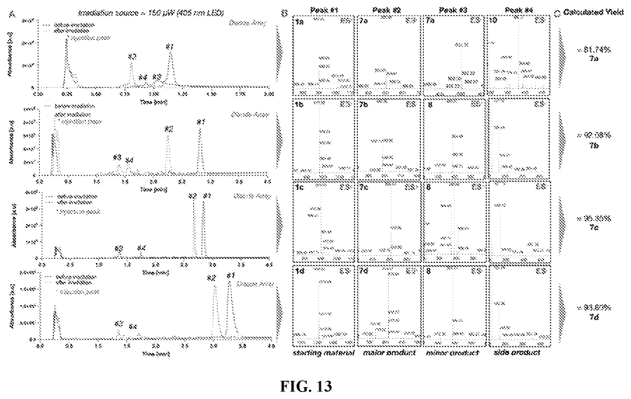

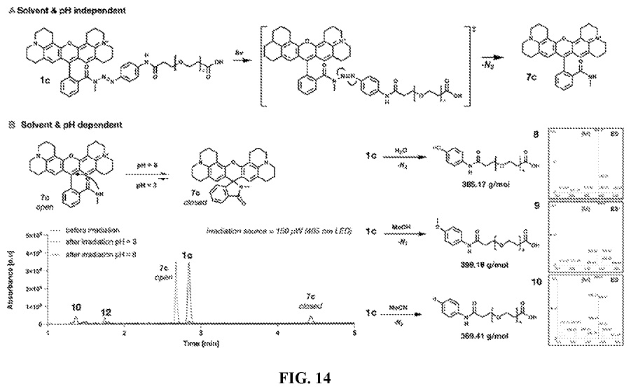

To understand the photophysical and photochemical behavior of the FLASH-off probes, spectroscopy was done in aqueous solutions containing probes 1a-d. All probes showed spectrally distinct fluorescence emission spectra ranging from 525-680 nm (FIG. 11). Their fluorescence intensity was examined across a wide pH range (pH=3-8), the fluorescent emission of FLASH-off probes was predominantly pH-independent (FIG. 12). In PBS, probes 1b and 1d were identified as the brightest analogs (Φ=73%, and 17%), and the most lipophilic analog 1c as the dimmest (Φ=3%, FIG. 9). From photolysis experiments using probes 1a-d and analysis by liquid chromatography coupled to mass spectrometry (LC/MS), the major photoproducts of the reaction were identified. Using mild irradiation conditions (405 nm LED, 150 ρW cm⁻²), the formation of the expected methyl xanthamide photoproducts 7a-d (FIG. 13) was observed exclusively. When photolysis was performed in an acidic buffer (pH=3), the open form of the xanthamide was detected (>95%). Irradiations performed in basic buffers (pH=8) revealed the presence of the closed spirocyclic isomer (>75%), as seen by their similar mass-to-charge ratio (582 m/z, electrospray ES, FIG. 3, B and FIG. 14). Conversely, phenol 8, anisole 9, and phenyl 10, analogs of the PEG4 linker, were detected when irradiating probes 1a-d in PBS, methanol, or acetonitrile respectively (FIG. 14). With these observations, it was proposed that the mechanism of photo-reaction has two components, a pH-independent photolytic cleavage with the release of N₂ followed by a pH- and solvent-dependent spirocyclization of the methyl xanthamide (FIGS. 3 and 14).

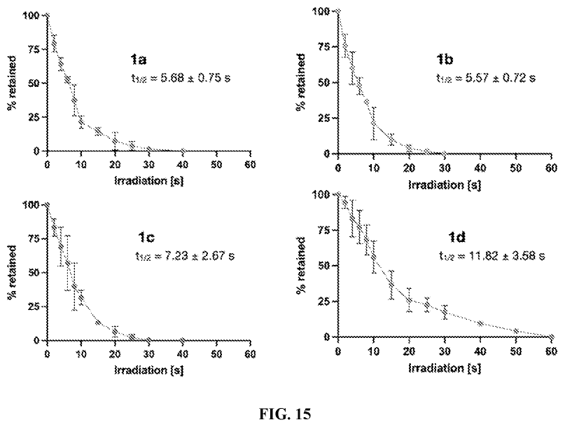

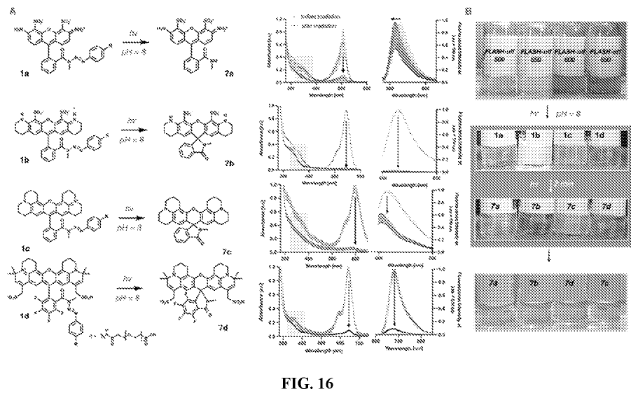

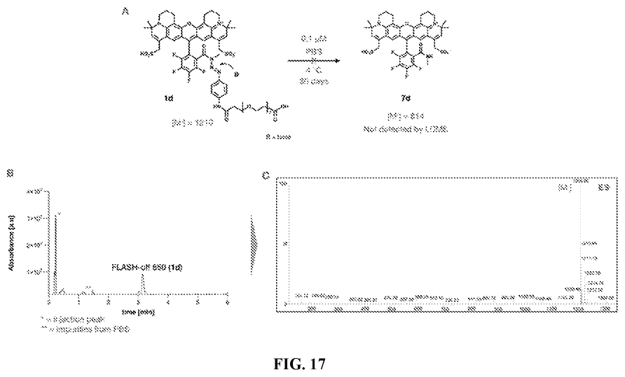

All photo reactions gave high yields of the methyl xanthamides photoproducts 7a-d (>90%, FIG. 13) and proceeded very rapidly with an average half-life of 5.67 s, 5.57 s, 7.23 s, and 11.82 s for 1a-d respectively (1.14 mW cm⁻², FIG. 15). Photolysis at pH=8 resulted in a 166-fold, 30-fold, 2-fold, and 0-fold decrease in fluorescence emission relative to compounds 1b, 1c, 1d, and 1a respectively (FIG. 3D and FIG. 16). After photolysis, a large decrease in absorbance and a hypsochromic shift (about 20 nm) were evident for all probes. This effect correlated with the loss of color for all solutions that underwent photolysis (FIG. 3, E and FIG. 16, B). The resistance of the triazene linker towards hydrolysis was tested in diluted PBS solutions (<1 μM). When stored at 4° C., no hydrolyzed adduct 7d was detected by LC/MS after 30 days. These results indicated that the cleavage of the triazene moiety proceeds selectively only after exposure to short UV/blue light irradiation and demonstrates the robust stability of probes 1a-d under physiological conditions required for live-cell imaging (FIG. 17). Due to their excellent reaction kinetics and spectroscopic behavior, N-hydroxysuccinimide (NHS) esters of probes 1b (FLASH-off 550) and 1d (FLASH-off 650) were made for constructing fluorescent antibody conjugates. Use of these conjugates was investigated as FLASH-off dyes for multiplexed imaging in live-, fixed cells, and human tissue.

### Example 4—Cellular Imaging

### Analysis of Fixed Immune Cells

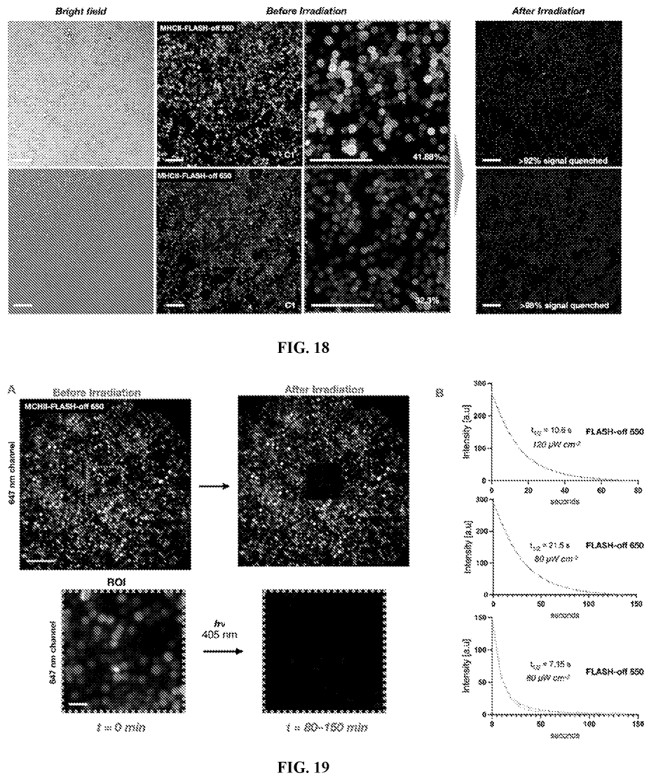

To determine the feasibility of antibody-FLASH-off conjugates, imaging of abundant immune cells (splenocytes) freshly harvested from mouse spleens was performed. The following was done: i) determining whether the different antibody-FLASH-off conjugates had similar morphologic appearances as conventional immunoconjugates, ii) how the quenching conditions translated to a microscope set-up, and iii) whether spectrally different FLASH-off probes resulted in the same staining pattern. Antigen-presenting cells were detected in a heterogeneous mixture of harvested splenocytes staining for the major histocompatibility complex (MHC) class II. Conjugates composed of anti-MHCII-FLASH-off 550 and anti-MHCII-FLASH-off 650 were incubated for 20 mins (5 μg/mL, DOL 3.4 and 4.0 respectively). Both probes showed good staining and yielded high-contrast images (SNR ˜8). Distinct circular staining patterns were observed that are indicative of the targeted MCHII membrane-bound proteins (FIG. 18). Similar percentages of labeled splenocytes were obtained using both FLASH-off 550 and FLASH-off 650 conjugates (41.88% and 32.2% respectively, FIG. 18). Complete quenching of the fluorescent signal (>92%) was achieved by short exposure to the 405 nm light (DAPI imaging channel, 4 s, 660 μW cm ⁻²). Quenching kinetics, however, could be adjusted by varying the light source (LED/laser) and power (FIG. 19). Exposure to constant light irradiation at imaging wavelengths indicated sustained photostability for both conjugates, especially when moderate LED/laser powers were used during image acquisition (<7 mW cm⁻²).

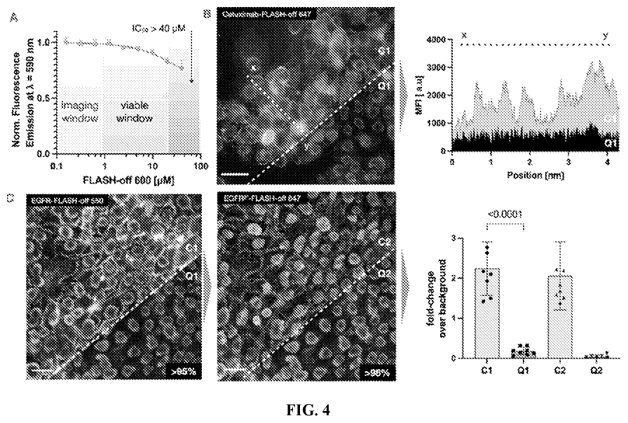

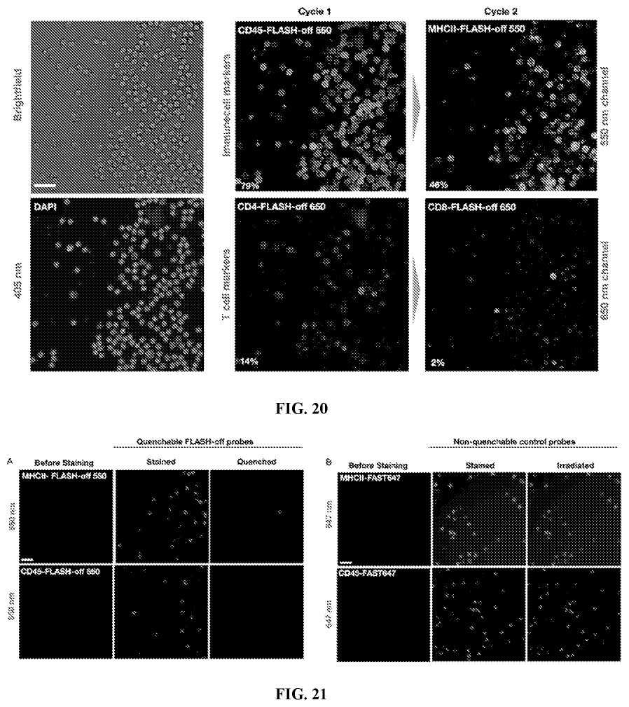

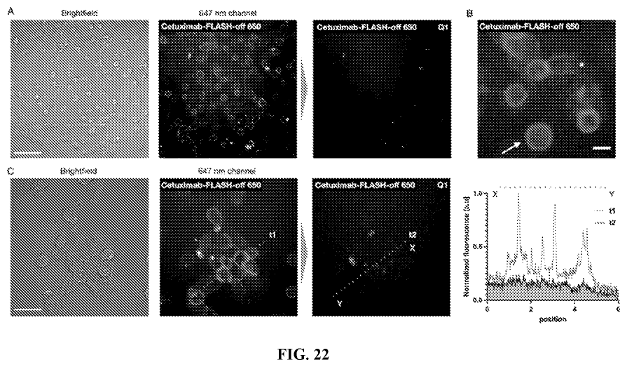

Cyclic imaging was performed during two cycles of staining to selectively distinguish four immune markers (CD45, MHCII, CD4, and CD8) and their abundance in splenocytes (FIG. 20). Statistical analysis of the total number of stained cells per marker matched previously reported values for murine splenocytes (79% CD45, 32% MHCII, 14% CD4 and 2% CD8 FIG. 20). Using the same imaging and quenching parameters, no signal reduction was observed in splenocytes stained with non-photoquenchable control (anti-MHCII-FAST647 and anti-CD45-FAST647, conjugates, FIG. 21). These results confirm that on-stage quenching is specific for FLASH-off probes because they are cleaved off from the antibody conjugate by an efficient photo-reaction and subsequently turned off by a fast spirocyclization that is favored at physiological pH. While the above experiments were done in fixed cells, FLASH-off probes were also applied to live cells (cultured epidermoid carcinoma A431) to determine i) toxicity and ii) live cell imaging capabilities. Using a presto blue viability assay of the membrane-permeable probe 1d, there was no apparent sign of cytotoxicity within 72 h of incubation at concentrations well above those used for imaging (<1 μM, FIG. 4A). Live A431 cells were next incubated with Cetuximab-FLASH-off 650 to stain for the epidermal growth factor receptor (EGFR). Distinctive membrane staining was observed, which co-localized with anti-EGFR-MB488 control. Importantly, complete signal quenching was achieved with sequential short pulses of UV light (4 s, FIG. 4, C and FIG. 22). Cyclic imaging was then tested using an anti-EGFR-FLASH-off 550 conjugate followed by a secondary antibody construct labeled with FLASH-off 650 (FIG. 4B). Staining cycles performed under 10 min showed good membrane staining (SNR ˜3), signal colocalization and optimal quenching (<90%, FIG. 4B). A live/dead indicator was used at the end of the third quenching cycle to confirm cell viability.

### Example 5—Multiplexed Tissue Imaging

Having shown the feasibility of fixed and live-cell imaging, cyclic imaging was studied in formalin-fixed paraffin-embedded (FFPE) sections as they represent one of the most common pathologic specimen types. Using human FFPE tonsil sections, we sought to i) identify the location and abundance of different immune markers, ii) determine whether FLASH-off probe staining was compatible with other non-photoquenchable probes, and iii) explore the limit of quenching resolution with enhanced local quenching control experiments.

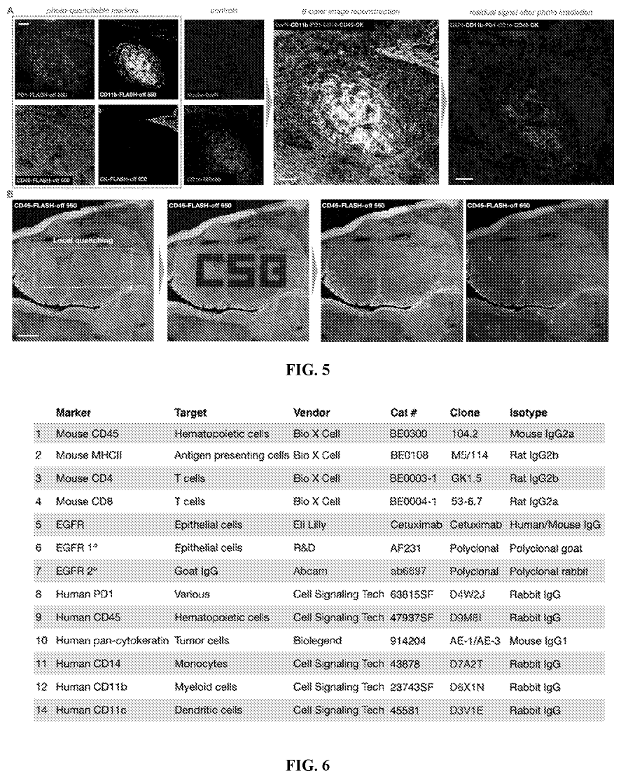

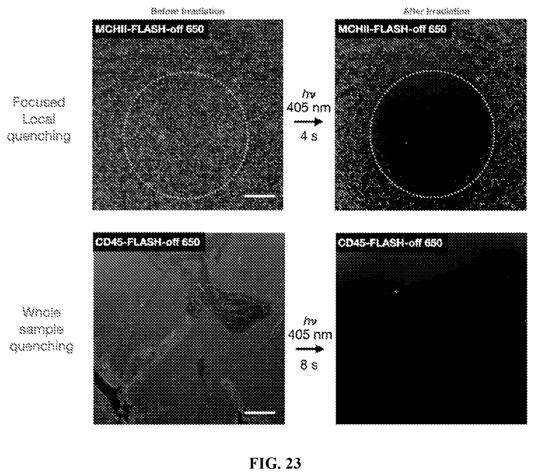

We prepared four antibody-FLASH-off conjugates that recognize human immune markers including CD45, CD11c, CD11b, and Pan-CK. Tonsil sections were stained with four FLASH-off antibody conjugates, one non-photoquenchable control CD14-MB488, and DAPI was used as a nuclear stain. Distinct staining patterns were observed for each antibody conjugate and compared the signal to previously validated staining patterns (FIG. 5A). UV light irradiation induced specific quenching of the fluorescent signal from the FLASH-off probes without causing an apparent decrease in signal intensity for the non-photo quenchable control or DAPI (FIG. 5A). Good spectral separation of all four channels allowed for the reconstruction of a field of view containing a total of six selected markers (FIG. 5A). Regions of high immune cell density were localized and the location of the selected immune markers was assessed within the distinctive morphological structures of tonsils tissue. To achieve quenching of the whole tonsil sample (>1 cm), the sample was irradiated with an external LED source (<2 min, 405 nm, power 1.14 mW×cm², FIG. 23).

Local quenching experiments were performed to test the limit of spatial resolution achievable after quenching FLASH-off antibody conjugates. Quenching was performed at a higher magnification (40×) relative to image acquisition (4×), instantly generating locally quenched spots within the stained sample (FIG. 23). Using a confocal scanning laser, unprecedented quenching control and resolution were demonstrated that allowed the writing of three letters “CSB” on a tissue section stained with anti-CD45-FLASH-off 550 and anti-CD45-FLASH-off 650 conjugates (FIG. 5B). Full signal recovery from the locally quenched region was achieved after incubation of the sample with a subsequent round of FLASH-off antibody conjugates (>18 h, 4° C., FIG. 5B).

### Discussion of Examples 1-5

The experimental results show that linear triazene linkers were used as a building block to create hybrid rhodamines which are bright and stable under imaging conditions but that can be “switched off” by a short pulse of UV light (405 nm, FIG. 1). The photochemical properties of, e.g., triazene-containing rhodamines 1a-d were examined with emission wavelength maxima between 520 and 680 nm. Probes 1b (FLASH-off 550) and 1d (FLASH-off 650) were selected to demonstrate fluorescence cyclic imaging without implementing any form of chemical handling (FIGS. 4-5, FIGS. 18-22). The discovery of controlled photo-quenchable probes advantageously allows for highly effective imaging and precision cycling.

Several alternative photo-immolative linkers (e.g., o-nitrobenzyl) able to release xanthamides after UV irradiation were examined. Even though the synthesis and photo reactions were feasible, the initial fluorescent state of the rhodamine-hybrids was completely quenched by the presence of the nitro groups that induce fluorescence quenching via photo-induced electron transfer (PET). Therefore, linear triazene was used. This linker advantageously can i) undergo fast photo-release kinetics, ii) produce non-toxic photoproducts, iii) resist hydrolysis under physiological conditions, and iv) be easily functionalized with water-soluble linkers. To enhance resistance toward spontaneous hydrolysis in aqueous buffer, two chemical approaches were used to decrease the nucleophilicity of the triazene rotamer. First, electron-withdrawing groups (e.g., N-aryl amides) were installed on the para position of the benzene ring, and second, the reactive primary amine was substituted with an acyl group formed during coupling to the rhodamine core (FIG. 1). Solution and cell experiments using FLASH-off probes demonstrated that this novel hybrid rhodamine system is efficient, fast, and compatible with live and fixed cell multiplexed cyclic imaging.

A number of additional features of the triazene-rhodamine system were noted. First, a bathochromic shift (about 20 nm) in the absorbance and fluorescence emission equally for all triazene-containing rhodamine analogs (FIG. 16). This feature is important for optimal spectroscopic characteristics of FLASH-off probes. Second, that the photolytic outcome of FLASH-off probes can be predicted by studying the photophysical behavior of the methyl xanthamide photoproducts 7a-d. Introducing aliphatic substituents on the anilines favored the formation of the dark, spirolactone xanthamides at pH >7. Without being bound by any particular theory or speculation, because probe 1a lacked these aliphatic substitutions, the spirocyclization of probe 7a did not proceed with increasing pH (FIG. 16). Increasing the water solubility of the triazene linker using, e.g., pegylated spacers allows biocompatible coupling to antibodies without affecting their function. However, spectroscopic studies on the non-sulfonated lipophilic probe 1c suggested that sulfonation of the xanthene core, in addition to pegylation, is important for generating bright, water-soluble, and biocompatible FLASH-off probes.

A number of different quenching technologies have been described in the literature. When comparing the different cyclic imaging methods, it is useful to ask three key questions: i) how much of the sample is destroyed or lost during repeated washing and generally harsh quenching conditions; ii) how fast is a given quenching step (often tens of minutes to hours); and iii) how fast or slow is each staining/destaining step?Most cycling methods were originally developed for paraffin-embedded tissue sections that can withstand harsh destaining conditions. Unfortunately, these harsh conditions require oxidants for bleaching and are not compatible with live-cell analysis. Furthermore, the early cycling technologies were slow and often required days of sample processing. The main advantages of the FLASH-off probe techniques include the minimal use of 405 nm light (1-10 s) for on-stage quenching without the need to perform any additional sample handling or washing steps. The experimental results provided in this disclosure show that the quenching kinetics are highly tunable and adaptable to both epifluorescent and confocal set-ups. In the local quenching control experiments, that quenching resolution solely depends on the scanning precision of the microscope, and it is equally efficient for FLASH-off 550 and FLASH-off 650 conjugates. The experimental data provided herein for rhodamine scaffolds can be extrapolated to fluorescein FLASH-off analogs, e.g., because their respective methyl xanthamides favor exclusively the non-emissive spirocyclic form, especially at low pH (FIG. 9). Thus, membrane-permeable fluorescein FLASH-off probes serve as complementary probes for targeting the intracellular domain of cells while allowing controlled quenching using light.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (I): 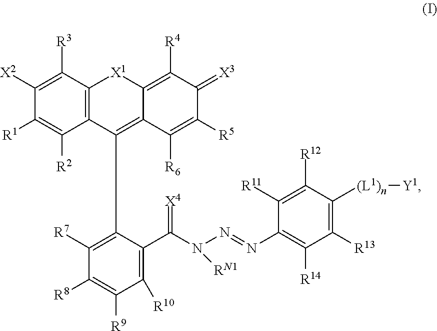
or a pharmaceutically acceptable salt thereof, wherein:
X₁ is selected from O, S, C(R₁₅)₂, and Si(R₁₅)₂;
each R₁₅ is independently selected from H, C₁₋₆ alkyl, C₂₋₆ alkenylene, and C₁₋₆ haloalkyl; wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃haloalkoxy;
or any two R¹⁵ together with the C or Si atom to which they are attached from a 3-7 membered saturated or unsaturated carbocyclic or heterocyclic ring, which is optionally substituted with 1 or 2 substituents independently selected from halo OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy;
R^{N1} is selected from C₁₋₃ alkyl and C₁₋₃ haloalkyl, wherein said C₁₋₃ alkyl is optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
X² is selected from OR^{N2} and N(R^{N2})₂;
X³ is selected from O and NR^{N2};
X⁴ is O, S; or NR^{N2};
each R^{N2} is independently selected from H, C₁₋₆ alkyl, and C₁₋₆ haloalkyl, wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
or any two R^{N2} together with the O or N atom to which they are attached from a 3-7 membered saturated or unsaturated carbocyclic or heterocyclic ring, which is optionally substituted with 1 or 2 substituents independently selected from halo, OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy;
R¹, R², R³, R⁴, R⁵, R⁶, R⁷, R⁸, R⁹, R¹⁰, R¹¹, R¹², R¹³, and R¹⁴ are each independently selected from H, halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C₁₋₆alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃ haloalkoxy;
or each pair of RN² and R³, R^{N2} and R¹, R^{N2} and R⁴, R^{N2} and R⁵, R¹ and R², and R⁵ and R⁶ together with the C, N, or O atoms to which they are attached, form a 5-8 membered saturated or unsaturated carbocyclic or heterocyclic ring, which is optionally substituted with 1, 2, or 3 substituents independently selected from halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C₁₋₆ alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃ haloalkoxy;
each L¹ is independently selected from N(R^{N}), O, C(═O), S(═O)₂, C₁₋₆ alkylene, —(OCH₂CH₂)ₓ—, and —(CH₂CH₂O) ₓ—;
n is an integer from 1 to 10;
each R^{N} is independently selected from H, C₁₋₃ alkyl, and C₁₋₃ haloalkyl;
each x is independently an integer from 1 to 2,000;
Y¹ is selected from NR^{N2}R^{1A}, OR^{2A}, C(═O)OR^{3A}, and a group reactive with a side chain of an amino acid of a protein.
R^{1A} selected from H and an amine protecting group;
R^{2A} is selected from H and an alcohol protecting group; and
R^{2A} is selected from H and a carboxylic acid protecting group.

2. The compound of claim 1, wherein the compound of Formula (I) has formula: 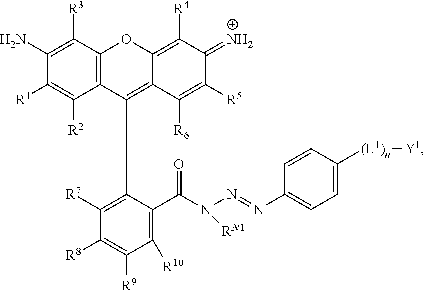
or a pharmaceutically acceptable salt thereof.

3. The compound of claim 2, wherein:
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino,
R⁷, R⁸, R⁹, and R¹⁰ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino; and
RN¹ is selected from C₁₋₃ alkyl and C₁₋₃ haloalkyl.

4. The compound of claim 3, wherein:
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, wherein said C₁₋₃ alkyl is optionally substituted with OH, SO₃H, C(═O)OH, CN, C₁₋₃ alkoxy, and C₁₋₃ haloalkoxy;
R⁷, R⁸, R⁹, and R¹⁰ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy; and
RN¹ is C₁₋₃ alkyl.

5. The compound of claim 1, wherein the compound of Formula (I) has formula: 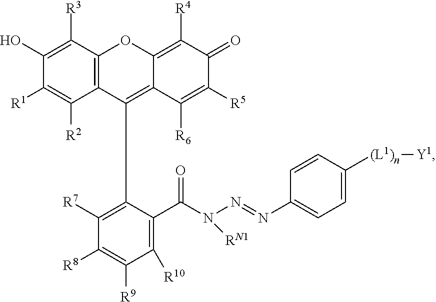
or a pharmaceutically acceptable salt thereof.

6. The compound of claim 5, wherein:
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino,
R⁷, R⁸, R⁹, and R¹⁰ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino; and
RN¹ is selected from C₁₋₃ alkyl and C₁₋₃ haloalkyl.

7. The compound of claim 6, wherein:
R¹, R², R³, R⁴, R⁵, and R⁶ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy, wherein said C₁₋₃ alkyl is optionally substituted with OH, SO₃H, C(═O)OH, CN, C₁₋₃ alkoxy, and C₁₋₃ haloalkoxy;
R⁷, R⁸, R⁹, and R¹⁰ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy; and
RN¹ is C₁₋₃ alkyl.

8. The compound of claim 1, wherein n is an integer from 1 to 5, and each L¹ is selected from NH, O, C(═O), and C₁₋₆ alkylene.

9. The compound of claim 1, selected from any one of the following compounds: 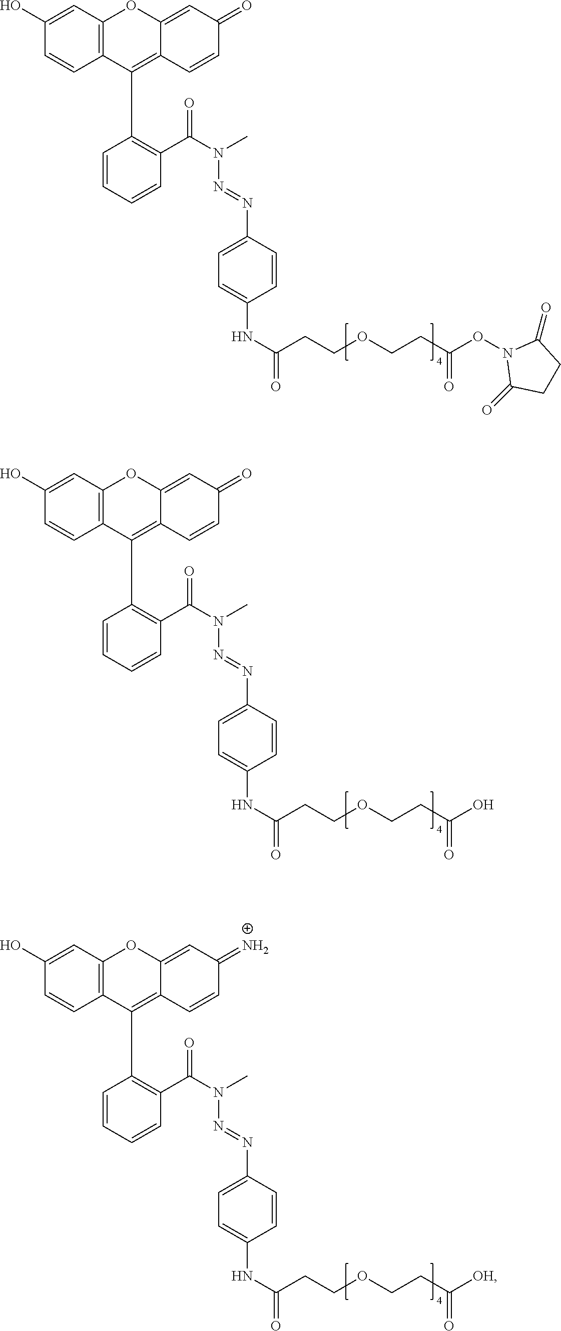 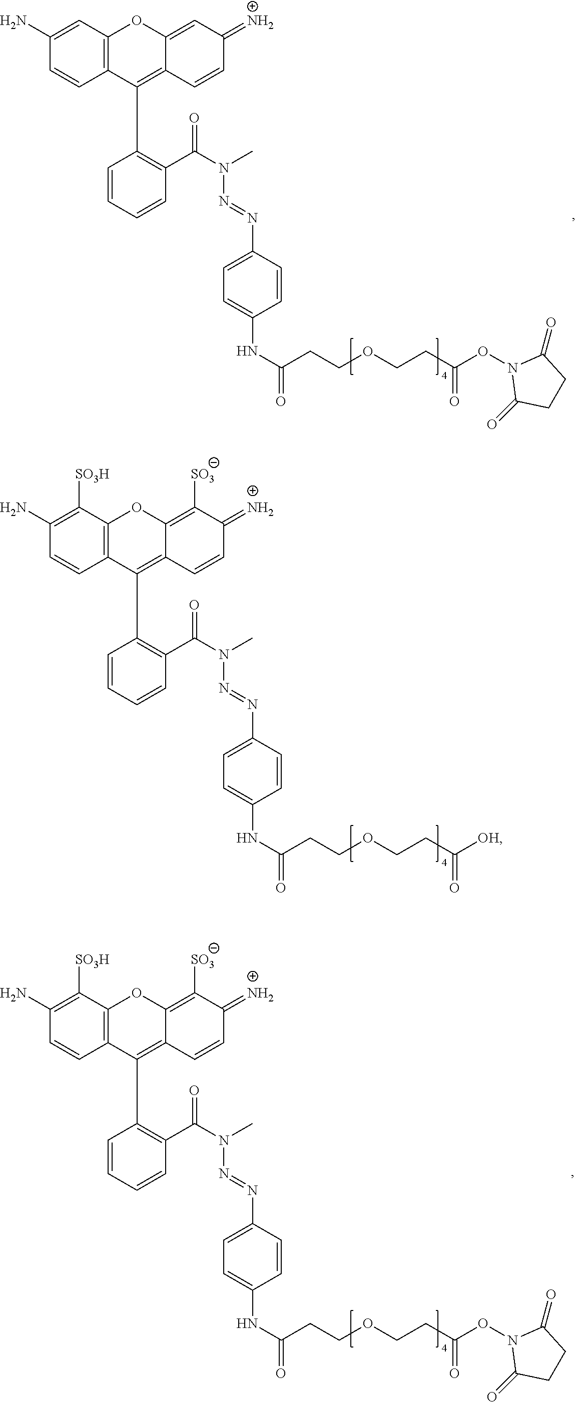 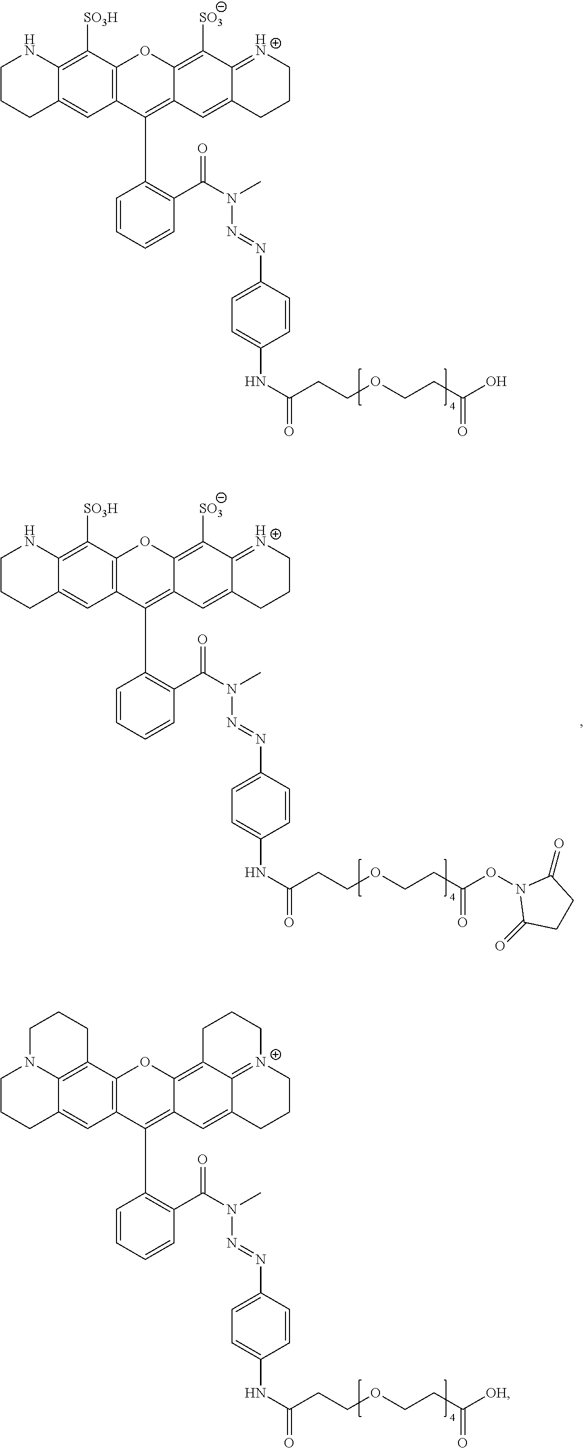 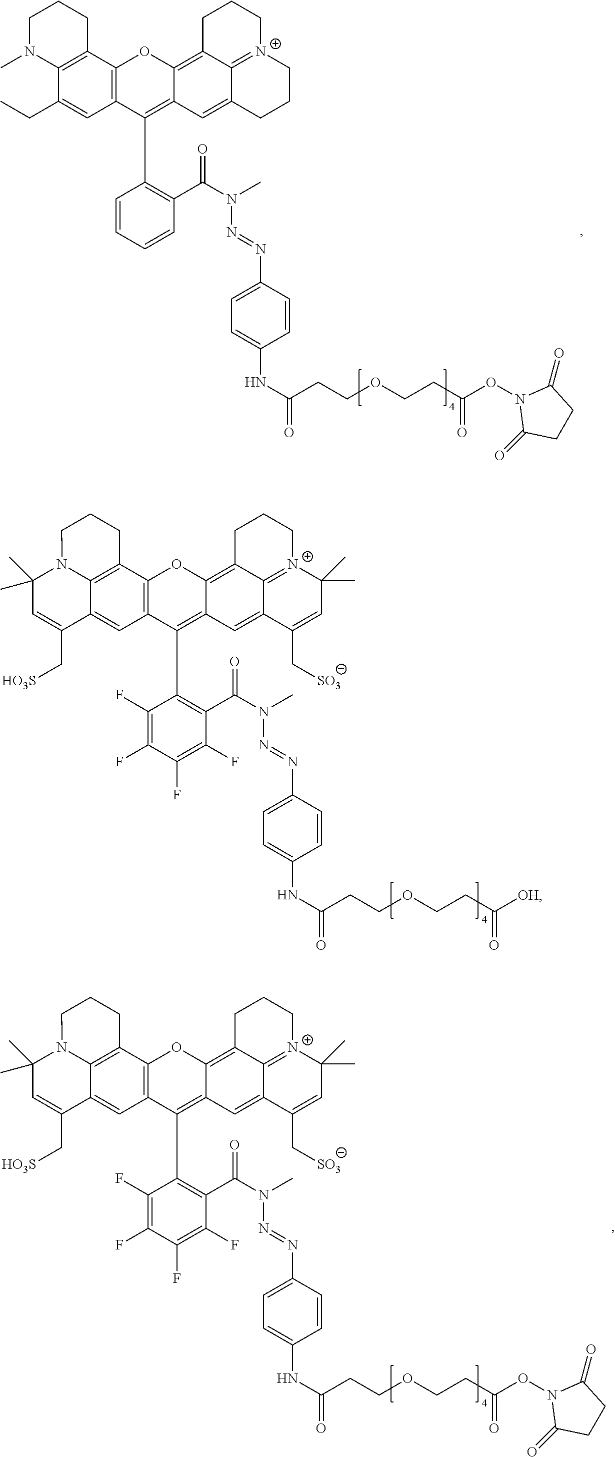 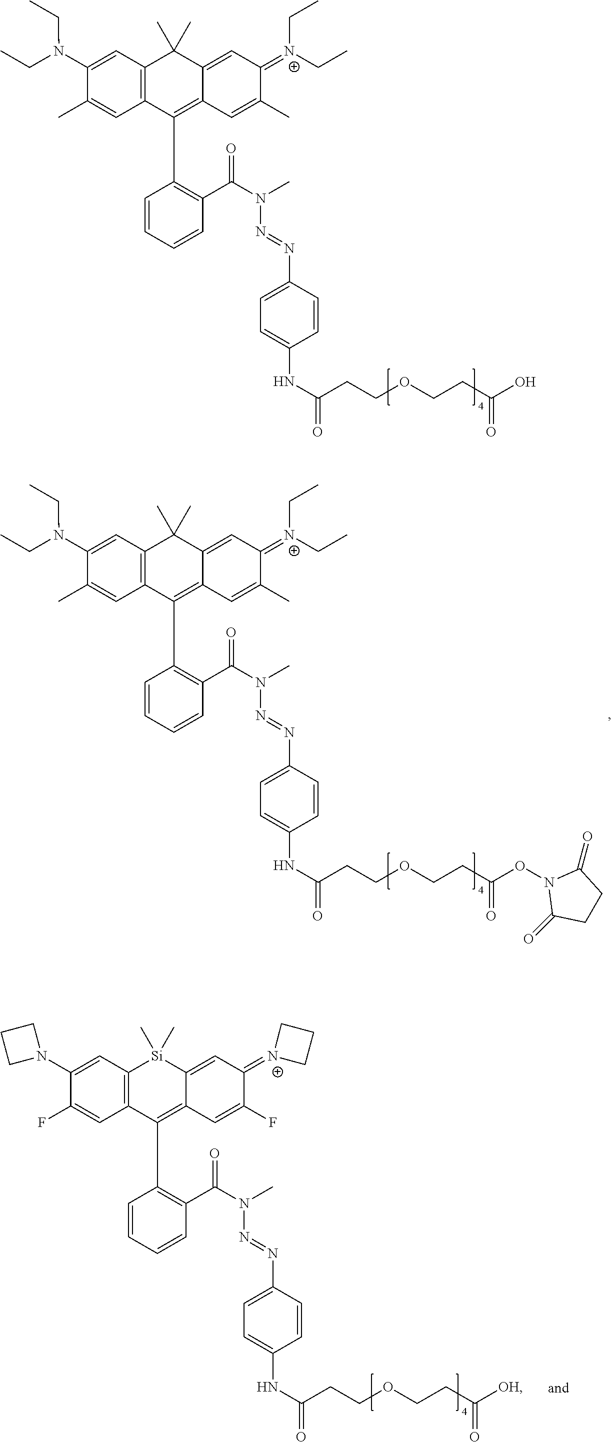 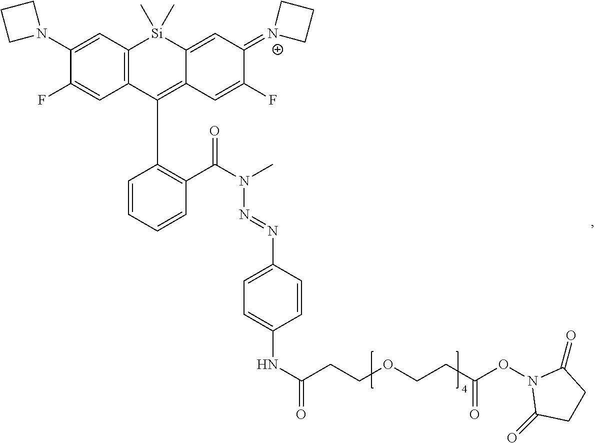
or a pharmaceutically acceptable salt thereof.

10. A protein conjugate of Formula (II): 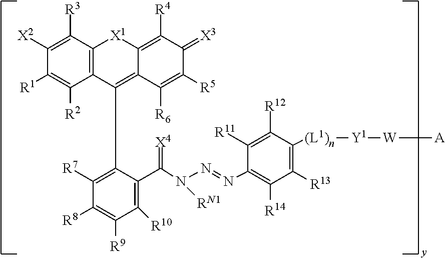
or a pharmaceutically acceptable salt thereof, wherein:
A is a protein;
y is an integer from 1 to 10;
Y¹ is a residue of a group which, prior to conjugation with the protein A, was a group reactive with a side chain of an amino acid of the protein A;
each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A;
X¹ is selected from O, S, C(R¹⁵)₂, and Si(R¹⁵)₂;
each R¹⁵ is independently selected from H, C₁₋₆ alkyl, C₂₋₆ alkenylene, and C₁₋₆ haloalkyl; wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
or any two R¹⁵ together with the C or Si atom to which they are attached from a 3-7 membered saturated or unsaturated carbocyclic or heterocyclic ring, which is optionally substituted with 1 or 2 substituents independently selected from halo OH, SH, NH₂, NO₂, CN, C₁₋₃ alkylamino, di(C₁₋₃alkyl)amino, C₁₋₃ alkylthio, C₁₋₃ alkoxy, and C₁₋₃ haloalkoxy;
R^{N1} is selected from C₁₋₃ alkyl and C₁₋₃ haloalkyl, wherein said C₁₋₃ alkyl is optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
X² is selected from OR^{N2} and N(R^{N2})₂;
X³ is selected from O and NR^{N2};
X⁴ is 0, S; or NR^{N2};
each R^{N2} is independently selected from H, C₁₋₆ alkyl, and C₁₋₆ haloalkyl, wherein said C₁₋₆ alkyl and C₁₋₆ haloalkyl are each optionally substituted with OH, SH, NH₂, NO₂, CN, C₁₋₃ alkylamino, di(C₁₋₃ alkyl)amino, C₁₋₃ alkylthio, C₁₋₃ alkoxy, or C₁₋₃ haloalkoxy;
or any two R^{N2} together with the O or N atom to which they are attached from a 3-7 membered saturated or unsaturated carbocyclic or heterocyclic ring, which is optionally substituted with 1 or 2 substituents independently selected from halo, OH, SH, NH₂, NO₂, CN, C₁₋₃ alkylamino, di(C₁₋₃ alkyl)amino, C₁₋₃ alkylthio, C₁₋₃ alkoxy, and C₁₋₃ haloalkoxy;
R¹, R², R³, R⁴, R⁵, R⁶, R⁷, R⁸, R⁹, R¹⁰, R¹¹, R¹², R¹³, and R¹⁴ are each independently selected from H, halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C1-6 alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃ haloalkoxy;
or each pair of R^{N2} and R³, R^{N2} and R¹, R^{N2} and R⁴, R^{N2} and R⁵, R¹ and R², and R⁵ and R^{6,} together with the C, N, or O atoms to which they are attached, form a 5-8 membered saturated or unsaturated carbocyclic or heterocyclic ring, which is optionally substituted with 1, 2, or 3 substituents independently selected from halo, OH, SH, SO₃H, NO₂, CN, C₁₋₆ alkyl, C₂₋₆ alkenyl, C₂₋₆ alkynyl, C₁₋₄ haloalkyl, C₁₋₆ alkoxy, C₁₋₆ haloalkoxy, amino, C₁₋₆ alkylamino, di(C₁₋₆ alkyl)amino, C₁₋₆ alkylthio, carbamyl, C₁₋₆ alkylcarbamyl, di(C₁₋₆ alkyl)carbamyl, and C(═O)OH, wherein said C₁₋₆ alkyl and C₁₋₄ haloalkyl are each optionally substituted with OH, SH, NH₂, NO₂, SO₃H, CN, C(═O)OH, C₁₋₃ alkylamino, di(C₁₋₃ alkyl)amino, C₁₋₃ alkylthio, C₁₋₃ alkoxy, or C₁₋₃haloalkoxy;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each R^{N} is independently selected from H, C₁₋₃ alkyl, and C₁₋₃ haloalkyl; and
each x is independently an integer from 1 to 2,000.

11. The conjugate of claim 10, wherein the protein is selected from an antibody, an antibody fragment, an engineered antibody, a peptide, and an aptamer.

12. The conjugate of claim 11, wherein the antibody is specific to an antigen which is a biomarker of a disease or condition.

13. The conjugate of claim 12, wherein the disease or condition is cancer.

14. The conjugate of claim 10, wherein Formula (II) has formula: 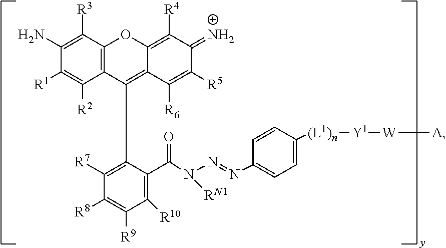
or a pharmaceutically acceptable salt thereof.

15. The conjugate of claim 10, wherein: y is
an integer from 4 to 6;
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino,
R⁷, R⁸, R⁹ and R¹⁰ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino; and
R^{N1} is selected from C₁₋₃ alkyl and C₁₋₃ haloalkyl.

16. The conjugate of claim 15, wherein:
R¹, R², R³, R⁴, R⁵, and R⁶ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy, wherein said C₁₋₃ alkyl is optionally substituted with OH, SO₃H, C(═O)OH, CN, C₁₋₃ alkoxy, and C₁₋₃ haloalkoxy;
R⁷, R⁸, R⁹ and R¹⁰ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy; and
R^{N1} is C₁₋₃ alkyl.

17. The conjugate of claim 10, wherein Formula (II) has formula: 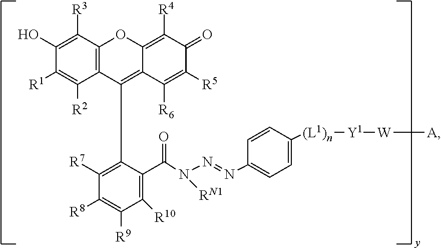
or a pharmaceutically acceptable salt thereof.

18. The conjugate of claim 17, wherein: y is an integer from 4 to 6;
R¹, R², R³, R⁴, R⁵, and R⁶ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino,
R⁷, R⁸, R⁹ and R¹⁰ are each independently selected from H, halo, OH, SH, SO₃H, C(═O)OH, NO₂, CN, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino, wherein said C₁₋₃ alkyl is optionally substituted with halo, OH, SH, SO₃H, C(═O)OH, NO₂, CN, C₁₋₃ alkoxy, C₁₋₃ haloalkoxy, amino, C₁₋₃ alkylamino, or di(C₁₋₃ alkyl)amino; and
R^{N1} is selected from C₁₋₃ alkyl and C₁₋₃ haloalkyl.

19. The conjugate of claim 18, wherein:
R¹, R², R³, R⁴, R⁵, and R⁶ are each independently selected from H, halo, OH, SO₃H, C(═O)OH, NO₂, CN, C₁₋₃ alkyl, C₁₋₃ haloalkyl, C₁₋₃ alkoxy, and C₁₋₃ haloalkoxy, wherein said C₁₋₃ alkyl is optionally substituted with OH, SO₃H, C(═O)OH, CN, C₁₋₃ alkoxy, and C₁₋₃ haloalkoxy;
R⁷, R⁸, R⁹ and R¹⁰ are each independently selected from H, halo, OH, SO₃H C(═O)OH, NO₂, CN, C₁₋₃ alkyl, C₁₋₃ haloalkyl, C₁₋₃ alkoxy, and C₁₋₃ haloalkoxy; and
R^{N1} is C₁₋₃ alkyl.

20. The conjugate of claim 10, wherein n is an integer from 1 to 5, and each L¹ is selected from NH, O, C(═O), and C₁₋₆ alkylene.

21. A composition comprising the conjugate of claim 10, or a pharmaceutically acceptable salt thereof, and an inert carrier.

22. A method of examining a cell or a component of a cell, the method comprising:
(i) contacting the cell with a conjugate of claim 10, or a pharmaceutically acceptable salt thereof;
(ii) imaging the cell with an imaging technique; and
(iii) after (ii), contacting the cell with a light of a wavelength.

23. The method of claim 22, wherein the wavelength is from about 350 nm to about 450 nm.

24. The method of claim 22, wherein the imaging technique is a fluorescence Imaging.

25. The method of claim 24, wherein the compound of Formula (II) has emission wavelength from about 500 nm to about 650 nm.

26. A method selected from:
profiling a cell;
examining a cell using a cytometry technique;
diagnosing a disease or condition of a subject by examining pathology of a cell obtained from the subject;
monitoring progression of disease or condition of a subject by examining pathology of a cell obtained from the subject; and
detecting a disease biomarker in a cell; the
method comprising:
(i) obtaining a cell from the subject; and
(ii) examining the cell according to the method of any one of paragraphs 22-25.

27. The method of claim 26, wherein the cell is obtained from the subject using image-guided biopsy, fine needle aspiration (FNA), surgical tissue harvesting, punch biopsy, liquid biopsy, brushing, swab, touch-prep, fluid aspiration or blood analysis.

28. The method of claim 26, wherein the cytometry technique is selected from image cytometry, holographic cytometry, Fourier ptychography cytometry, and fluorescence cytometry.

29. The method of claim 26, wherein the cell is selected from a cancer cell, an immune system cell, and a host cell.

30. The method of claim 29, wherein the disease or condition is cancer.
